(19) United States
(12) Patent Application Publication
(10) Pub. No.: US 2012/0209110 A1
(43) Pub. Date: Aug. 16, 2012

(54) Optimized Placement of Cannula for Delivery of Therapeutics to the Brain

(76) Inventors: Krystof S. Bankiewicz, Oakland, CA (US); Dali Yin, South San Francisco, CA (US)

(21) Appl. No.: 13/391,606

(22) PCT Filed: Aug. 25, 2010
(86) PCT No.: PCT/US10/46680

§ 371 Date: Apr. 25, 2012

Related U.S. Application Data
(60) Provisional application No. 61/275,209, filed on Aug. 25, 2009.

(51) Int. Cl. A61M 5/00; A61B 6/00

(52) U.S. Cl. 600/431; 604/506

(57) ABSTRACT

Methods and systems are provided for improved delivery of agents to targeted regions of the brain, by the use of placement coordinates that provide for optimal placement of delivery cannula. By optimizing the cannula placement, reproducible distribution of infusate in the targeted region of the brain is achieved, allowing a more effective delivery of therapeutics to the brain.

### BACKGROUND OF THE INVENTION

Convection-enhanced delivery (CED) is an interstitial central nervous system (CNS) delivery technique that also circumvents the blood-brain barrier in delivering agents into the central nervous system (CNS). Traditional local delivery of most therapeutic agents into the brain has relied on diffusion, which depends on a concentration gradient. The rate of diffusion is inversely proportional to the size of the agent, and is usually slow with respect to tissue clearance. Thus, diffusion results in a non-homogeneous distribution of most delivered agents and is restricted to a few millimeters from the source. In contrast, CED uses a fluid pressure gradient established at the tip of an infusion catheter and bulk flow to propagate substances within the extracellular fluid space. CED allows the extracellularly-infused material to further propagate via the perivascular spaces and the rhythmic contractions of blood vessels acting as an efficient motive force for the infusate. As a result, a higher concentration of drug is distributed more evenly over a larger area of targeted tissue than would be seen with a simple injection. Currently, CED has been clinically tested in the fields of neurodegenerative diseases, such as Parkinson's disease (PD), and neuro-oncology. Laboratory investigations with CED cover a broad field of application, such as the delivery of small molecules, macromolecules, viral particles, magnetic nanoparticles, and liposomes.

CED visualization with the aid of novel contrast materials co-infused with therapeutic agents has been investigated in rodent, non-human primates (NHP) and humans. During CED, the volume of distribution (Vd) for a given agent depends on the structural properties of the tissue being convected, such as hydraulic conductivity, vascular volume fraction, and extracellular fluid fraction. It also depends on the technical parameters of infusion procedure such as cannula design, cannula placement, infusion volume, and rate of infusion to improve delivery efficiency while attempting to limit the spread of the therapeutic into regions outside the target.

Image-guided neuronavigation utilizes the principle of stereotaxis. The brain is considered as a geometric volume which can be divided by three imaginary intersecting spatial planes, orthogonal to each other (horizontal, frontal and sagittal) based on the Cartesian coordinate system. Any point within the brain can be specified by measuring its distance along these three intersecting planes. Neuronavigation provides a precise surgical guidance by referencing this coordinate system of the brain with a parallel coordinate system of the three-dimensional image data of the patient that is displayed on the console of the computer-workstation so that the medical images become point-to-point maps of the corresponding actual locations within the brain (see Golfinos et al. J Neurosurg 1995; 83:197-205). The integration of functional imaging modalities, in particular, the magnetoencephalography (MEG), functional magnetic resonance imaging (fMRI) and positron emission tomography (PET) with neuronavigation has permitted significant advances in neurology.

The present invention provides improved methods for cannula placement.

### SUMMARY OF THE INVENTION

Methods and systems are provided for improved delivery of therapeutic agents to targeted regions of the brain, by the positioning of the delivery cannula to provide for optimal placement. The guidelines for cannula positioning of the invention avoid delivery of a therapeutic agent to “leakage pathways” present in the brain, and by utilizing the guidelines for cannula placement, reproducible distribution of infusate in the targeted region of the brain is achieved, allowing a more effective delivery of therapeutics to the brain. Usually it is preferred that a leakage pathway be greater than 1 mm distance from a delivery tip. Regions of interest for targeting include, without limitation, putamen, thalamus, brain stem, etc. In some embodiments, the recipient is a primate, e.g. humans and non-human primates.

Methods are also provided for determining optimal positioning for cannula placement. In some embodiments the placement is determined experimentally, by the method of: delivering an imaging agent to the targeted region of the brain, determining the distribution of the infusate; and correlating the site of cannula placement with the desired distribution, wherein the optimal placement results in appropriately contained infusate, i.e. the infusate does not spread outside of the desired target area. In other embodiments, the placement positioning provided herein is used to extrapolate from one species to another, through 3 dimensional modeling techniques.

Systems are provided for delivery of therapeutic agents to the brain, where the system comprises a delivery cannula, and a stereotactic system provided with the placement coordinates for optimal cannula placement.

The administration of therapeutic agents of the present invention can be via any localized delivery system that allows for the delivery of a therapeutic agent. Examples of such delivery systems include, but are not limited to CED, and intracerebral delivery, particularly CED.

In some embodiments of the invention, the delivery cannula is a step-design cannula, which reduces the reflux along the infusion device by restricting initial backflow of fluid flow beyond the step. In such methods, the placement coordinates of the invention allow optimal site of placement of the step and/or tip of the infusion cannula within targeted tissue in a manner that avoids delivery of a therapeutic agent to leakage pathways in the brain, such as surrounding white matter tracts, blood vessels, ventricles, and the like that act as leakage pathways in the brain.

In one aspect, the invention provides methods for treating a patient having a CNS disorder characterized by neuronal death and/or dysfunction. In one embodiment, the CNS disorder is a chronic disorder. In another embodiment, the CNS disorder is an acute disorder. CNS disorders of interest for treatment by the methods of the invention include, without limitation, Huntington's disease, Alzheimer's disease, amyotrophic lateral sclerosis (ALS), Parkinson's disease, stroke, head trauma, spinal cord injury, multiple sclerosis, dementia with Lewy Bodies, retinal degeneration, epilepsy, psychiatric disorders, disorders of hormonal balance, and cochlear degeneration. Treatment methods may include prophylactic methods, e.g. involving preoperative diagnosis. Preoperative diagnosis may include, without limitation, genetic screening; neuroimaging; etc. Neuroimaging may comprise functional neuroimaging or non-functional imaging, e.g. PET, MRI, and/or CT.

In another aspect, the invention provides prophylactic methods for treating a patient at risk for a CNS disorder. The methods comprise locally delivering a pharmaceutical composition to a responsive CNS neuronal population in the patient utilizing the cannula placement coordinates of the present invention, wherein such administration of the growth factor prevents or delays onset of a CNS disorder, or reduces the severity of the CNS disorder once it is manifest.

These and other aspects and embodiments of the invention and methods for making and using the invention are described in more detail in the description of the drawings and the invention, the examples, the claims, and the drawings that follow.

### BRIEF DESCRIPTION OF THE DRAWINGS

FIG. 1: Correlation of spatial coordinates and length of backflow with distribution of MRI tracer in the putamen.

FIG. 2: (A) Schematic of the step cannula placement in the putamen. Both step and tip portion of the cannula placement in green, blue and red zone for each case are shown. (B) Success of distribution defined as Vd in putamen vs. total Vd for each zone is shown (p<0.01). (C). Representative MR images showing distribution of Gadoteridol in the putamen for green, blue and red zone. Cannula placement and initial infusion are shown in panels C, D and E for each zone. Panels F, G and H show distribution of Gadoteridol in the brain after infusion into respective RGB zones. Note minimal leakage into white matter tracts in G (blue) and pronounced leakage in H (red). Infusion into green zone (F) resulted in tracer distribution in putamen only.

FIG. 3: RGB zones for step outlined in the putamen of NHP (A) and human putamen (B) based on the RGB parameters obtained in the NHP and compared using the same scale.

FIG. 4: 3D reconstruction of green zone and representative volumes of “green zone” in NHP (A and C) and human putamen (B and D). Area of green zone was defined from MR images as a volume at least 3 mm ventral to the CC, at least 6 mm away from the AC (3 mm from cannula tip to AC plus 3 mm of tip length) vertically, greater than 2.75 mm from EC laterally, and more than 3 mm from IC medially.

FIG. 5: Representative MR images showing distribution of Gadoteridol in the putamen and leakage into white matter tract at small and large infusion volume of MRI tracer.

FIG. 6 shows the percent of Vd of Gd in the thalamus vs total Vd in thalamus and WMT.

FIG. 7 shows cannula placement in the thalamus.

FIG. 8 percent of infused tracer contained within the thalamus is plotted against entry point.

FIG. 9 percent of infused tracer contained within the thalamus is plotted against lateral border.

FIG. 10. The distance from the cannula step to midline correlated with thalamus containment.

FIG. 11. Distribution of Gadoteridol in the brainstem during CED.

FIG. 12. Measurements of parameters for cannula step placement in the brainstem.

FIG. 13 shows brain stem containment against measured parameters.

FIG. 14 shows Vi versus Vd in thalamus and brainstem.

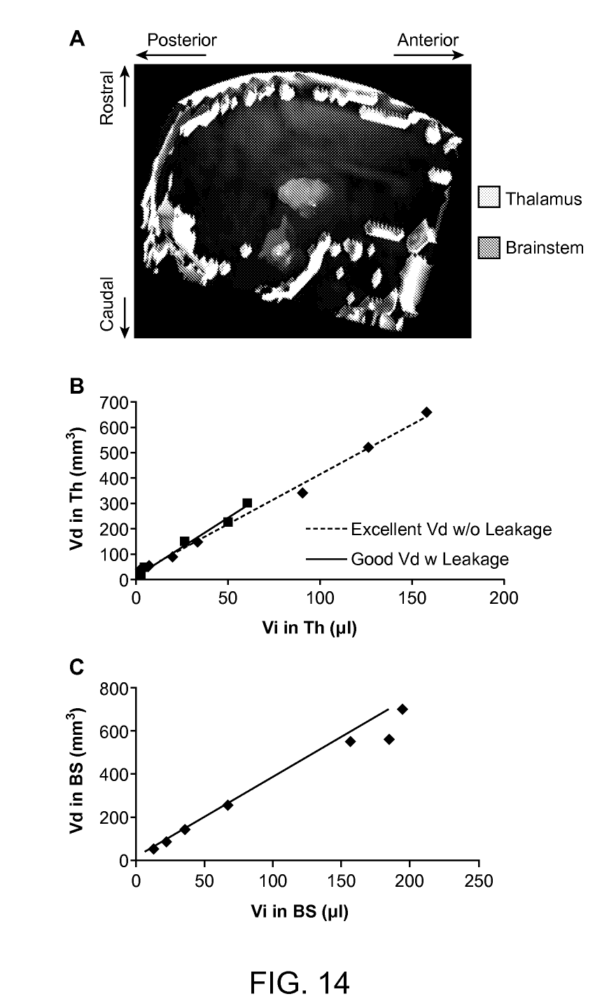

FIG. 15. T1-weighted MR images with Gd RCD and 3D construction of ROI. (a)-(e) are a series of real-time T1-weighted MR images in the coronal plane obtained at various time point from the beginning to the end of infusion into the thalamus of a NHP. The volume of infusate (Vᵢ) at the corresponding infusion time point is indicated at the bottom of each panel. Scale bar=0.5 cm. (f) shows a 3D reconstruction of ROI based on Gd signal in the left thalamus after infusion finished. The volume of Gd distribution (V_{d}) is indicated at the bottom of the panel. RCD: real-time convective delivery. ROI: region of interest.

FIG. 16. Linear relationship between Vᵢ and V_{d} in NPH infused with AAV2-GDNF/Gd. Plot shows a linear relationship (R²=0.904, P<0.0001) between Vᵢ and V_{d} in NHP (n=5). The mean V_{d}/Vᵢ ratio was 4.68±0.33 (mean±SEM). Vᵢ: infusate volume. V_{d}: distribution volume of Gd.

FIG. 17. MRI correlation with histology in primate #1 with bilateral infusion of AAV2-GDNF into the thalamus. (a). T1-weighted MR image showing Gd distribution in the thalamus, outlined in green. Areas staining positive for GDNF (outlined in orange) of corresponding histologic sections were transferred to the MR image for comparison. Since the left and right infusions were completed by different times, the final series of MR images for each infusion was cropped and merged in panel a. Infusion volume to the left and right brain was indicated at the bottom of the panel [Vᵢ(L) and Vᵢ(R)]. Scale bar=0.5 cm. (b). Coronal histologic section of primate brain imaged in a, showing GDNF staining in a pattern similar to that noted on MRI with Gd. Scale bar=1 cm. (c) High magnification of boxed insert in b, showing GDNF-positive cells within the thalamus. Scale bar=50 mm. (d) and (e) show the areas of Gd distribution and GDNF expression on the left (d) and right (e) side of the brain in a series of MR images. r: correlation coefficient.

FIG. 18. MRI correlation with histology in primate #2 with unilateral co-infusion of AAV2-GDNF and AAV2-AADC into the thalamus. (a) T1-weighted MR image showing Gd distribution in the thalamus, outlined in green. Areas staining positive for GDNF (outlined in orange) and AADC (outlined in blue) of corresponding histologic sections were transferred to the MR image for comparison. Scale bar=0.5 cm. (b) Coronal histologic section of primate brain imaged in a, showing GDNF staining in a pattern similar to that noted on MRI with Gd. Scale bar=1 cm. (c) AADC stained histologic section adjacent to b, showing both endogenous and transduced AADC expression. Transduced AADC were outlined in blue. (e) AADC and TH co-labeled histologic section adjacent to c, showing co-staining for AADC in brown and tyrosine hydroxylase (TH) in red to differentiate endogenous AADC/TH (in dark red) from transduced AADC (in brown). The expression pattern of transduced AADC is nearly identical to GDNF expression in b. (e) High magnification of boxed insert in c showing endogenous AADC-positive cells in the nigra. Scale bar=200 mm. (f) High magnification of boxed insert in d showing AADC/TH-positive cells in the nigra. Scale bar=200 mm. (g) High magnification of boxed insert in c showing endogenous AADC-positive fibers in the putamen. Scale bar=200 mm. (h) High magnification of boxed insert in c showing AADC-positive cells in the putamen. Scale bar=200 mm. (i) high magnification of boxed insert in d showing AADC-positive cells in the thalamus. Scale bar=200 mm. (j) shows the areas of Gd, GDNF and AADC distribution on the right side of the brain in a series of MR images. r₁: correlation coefficient between areas of Gd and GDNF expression. r₂: correlation coefficient between areas of Gd and AADC expression. r₃: correlation coefficient between areas of GDNF and AADC expression.

FIG. 19. MRI correlation with histology in primate #3 with bilateral co-infusion of AAV2-GDNF and AAV2-AADC into the thalamus. (a) T1-weighted MR image showing Gd distribution in the thalamus, outlined in green. Areas staining positive for GDNF (outlined in orange) and AADC (outlined in blue) of corresponding histologic sections were transferred to the MR image for comparison. Scale bar=0.5 cm. (b) Coronal histologic section of primate brain imaged in a, showing GDNF staining in a pattern similar to that noted on MRI with Gd. Scale bar=1 cm. (c) AADC and TH co-labeled histologic section adjacent to b, showing co-staining for AADC in brown and tyrosine hydroxylase (TH) in red. (d) and (e) show the areas of Gd, GDNF and AADC distribution on the left (d) and right (e) side of the brain in a series of MR images. r₁: correlation coefficient between areas of Gd and GDNF expression. r₂: correlation coefficient between areas of Gd and AADC expression. r₃: correlation coefficient between areas of GDNF and AADC expression.

FIGS. 20A-D. Failure of the CED due to cannula tip placement outside of the “Green Zone”. A. Cannula tip is placed too close to leakage pathway (axonal track) leading to infusion into the anterior commissure (B) rather than to the putamen. C. Cannula tip is placed too close to leakage pathway (blood vessel) leading to infusion into the perivascular space (D) rather than to the putamen.

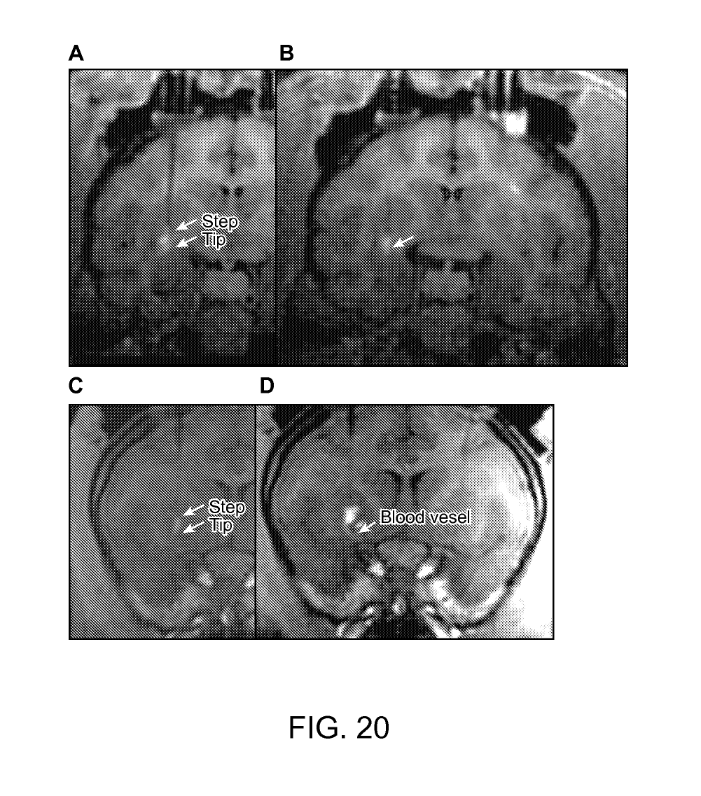

### DETAILED DESCRIPTION OF THE EMBODIMENTS

Optimal results in the direct brain delivery of brain therapeutics, such as proteins, including growth factors, polynucleotides, viral vectors, etc. into primate brain depend on reproducible distribution throughout the target region. Provided herein are placement coordinates that define an optimal site for infusions into non-human primate and human brains for targeted regions, which placement coordinates allow the avoidance of leakage pathways in the brain, e.g. by positioning at least 1 mm, at least 1.5 mm, at least 2 mm or more distance between delivery tip and leakage pathway.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an individual” includes one or more individuals and reference to “the method” includes reference to equivalent steps and methods known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Stereotactic Delivery: A computer-based modality for exact placement of points in the brain. Stereotactic methods may utilize a brain atlas, a number of which are available in digital form. For example the Talairach-Tournoux (TT) atlas (see Nowinski (2005) Neuroinformatics 3:293-300 for a review) is available in electronic format. The atlas provides a 3 dimensional representation of the brain for fast and automatic interpretation of images.

Stereotactic delivery may use a frame, in which a frame is attached to the skull to provide a fixed reference point. This point, combined with a three-dimensional image of the brain provided by a computer and MRI scanning, allows for precise mapping and visualization of the targeted region. Precise navigation to the target site is possible using a variety of devices attached to the frame. Alternatively, frameless stereotactic delivery provides precision of placement by substituting a frame for a reference system created by “wands,” plastic guides, or infrared markers.

Functional MRI (fMRI) may be used to pinpoint functional areas of the brain. While the MRI is scanning, the patient is asked to perform a series of activities and movements, such as reading a list or tapping fingers. The areas of the brain that correlate to these movements and activities “light up” on the scan and create an image. This information is used by surgical navigation computers in the planning of incisions, skull openings and tumor removal to minimize neurological deficits. Computed tomography (CT) is a scanning tool that combines X-ray with a computer to produce detailed images of the brain.

Imaging. The in vivo distribution of an infusate may be determined with imaging where a molecule with a detectable label is infused to the target region of the brain, and the spread through the brain determined by MRI, positron emission tomography (PET), etc. Suitable labels for the selected tracer include any composition detectable by spectroscopic, photochemical, immunochemical, electrical, optical or chemical means. Useful labels in the present invention include radiolabels, e.g. ¹⁸F, ³H, ¹²⁵I, ³⁵S, ³²P, etc), enzymes, colorimetric labels, fluorescent dyes, and the like. Means of detecting labels are well know to those of skill in the art. For example, radiolabels may be detected using imaging techniques, photographic film or scintillation counters. In some embodiments liposomes are labeled, e.g. with Gadoteridol, for imaging by MRI.

Reference coordinates. The X, Y and Z axial values of cannula placement is determined by imaging, e.g. magnetic resonance imaging, where MR images are projected in all three dimensions (axial, coronal and sagittal). For convenience and in accordance with conventional methods, the midpoint of the anterior commissure-posterior commissure (AC-PC) line may be designated as zero point (0,0,0) of three-dimensional (3D) brain space. The AC-PC line goes from the superior surface of the anterior commissure to the center of the posterior commissure. After determining the AC-PC line on midsagittal plane of MRI, the midpoint of AC-PC line may be determined. Using the horizontal and vertical plane through the midpoint of AC-PC line, all three planes can be displayed, and the X, Y and Z axial values of cannula position can be obtained by measurements of distance from cannula to midline on coronal MRI plane (X value), distance anterior (or posterior) to the midpoint of AC-PC line of the coronal MRI plane (Y value), and the distance above (or below) axial plane incorporating the AC-PC line on MRI (Z value).

Leakage pathways. As used herein, the term “leakage pathway” refers to physical structures in the central nervous system, particularly in the brain, that transport soluble agents. When therapeutic agents are delivered to tissues in close proximity of such leakage pathways, the agent may be adversely transported to non-targeted regions. Anatomic structures that provide for leakage pathways in the CNS include, without limitation, axon tracts, blood vessels, perivascular spaces, and ventricular spaces.

Blood-Brain Barrier: A wall of nerves and cells surrounding the brain membrane. While this barrier has a protective function, it also reduces the ability of therapeutic drugs to effectively reach targeted regions of the brain.

Putamen: a round structure located at the base of the forebrain (telencephalon). The putamen and caudate nucleus together form the dorsal striatum. It is also one of the structures that comprises the basal ganglia. Through various pathways, the putamen is connected to the substantia nigra and globus pallidus. The main function of the putamen is to regulate movements and influence various types of learning. It employs dopamine to perform its functions. The putamen also plays a role in degenerative neurological disorders, such as Parkinson's disease.

Brain stem: The brain stem, located at the front of the cerebellum, links the cerebrum to the spinal cord and controls various automatic as well as motor functions. It is composed of the medulla oblongata, the pons, the midbrain, and the reticular formation.

Cerebellum: Located at the back of the brain, the cerebellum controls body movement, i.e., balance, walking, etc.

Cerebrum: The brain's largest section can be divided into two parts: the left and right cerebral hemispheres. These hemispheres are joined by the corpus callosum, which enables “messages” to be delivered between the two halves. The right side of the brain controls the left side of the body, and vice versa. Each hemisphere also has four lobes that are responsible for different functions: frontal; temporal; parieta, and occipital.

Cranium: The bony covering that surrounds the brain. The cranium and the facial bones comprise the skull.

Hypothalamus: The part of the brain that acts as a messenger to the pituitary gland; it also plays an integral role in body temperature, sleep, appetite, and sexual behavior.

Midbrain: Part of the brain stem, it is the origin of the third and fourth cranial nerves which control eye movement and eyelid opening.

Pons: This part of the brain stem is the origin of four pairs of cranial nerves: fifth (facial sensation); sixth (eye movement); seventh (taste, facial expression, eyelid closure); and eighth (hearing and balance).

Posterior fossa: The part of the skull containing the brain stem and the cerebellum.

Thalamus: A small area in the brain that relays information to and from the cortex.

Primates. A primate is a member of the biological order Primates, the group that contains lemurs, the Aye-aye, lorisids, galagos, tarsiers, monkeys, and apes, with the last category including great apes. Primates are divided into prosimians and simians, where simians include monkeys and apes. Simians are divided into two groups: the platyrrhines or New World monkeys and the catarrhine monkeys of Africa and southeastern Asia. The New World monkeys include the capuchin, howler and squirrel monkeys, and the catarrhines include the Old World monkeys such as baboons and macaques and the apes.

The methods of the invention are applicable to all primates. Of particular interest are simians. In some embodiments the methods are applied to humans. In other embodiments the methods are applied to non-human primates.

Assessing includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

As used herein, “treatment” or “treating” refers to inhibiting the progression of a disease or disorder, or delaying the onset of a disease or disorder, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect may be prophylactic in terms of completely or partially preventing a disease or condition, or a symptom thereof and/or may be therapeutic in terms of a partial or complete cure for a disease or disorder and/or adverse affect attributable to the disease or disorder. “Treatment,” as used herein, covers any treatment of a disease or disorder in a mammal, such as a human, and includes: decreasing the risk of death due to the disease; preventing the disease of disorder from occurring in a subject which may be predisposed to the disease but has not yet been diagnosed as having it; inhibiting the disease or disorder, i.e., arresting its development (e.g., reducing the rate of disease progression); and relieving the disease, i.e., causing regression of the disease. Therapeutic benefits of the present invention include, but are not necessarily limited to, reduction of risk of onset or severity of disease or conditions associated with Parkinson's disease.

Delivery cannula. The methods of the invention allow for accurate placement of any delivery cannula, as are known in the art. For example, see the reviews inter alia, herein specifically incorporated by reference: Fiandaca et al. (2008) Neurotherapeutics. 5(1):123-7; Hunter et al. (2004) Radiographics 24(1):257-85; and Ommaya (1984) Cancer Drug Deliv. 1(2):169-79.

Delivery cannula of particular interest step design reflux resistant cannula, which find particular use in convection-enhanced delivery (CED). Such cannulas are described, for example, by Krauze et al. (2005) J. Neurosurg. 103(5):923-9; and in the published patent applications US 2007-0088295; and US 2006-0135945, each of which is specifically incorporated by reference.

Reference may be made herein to the placement of a reflux-resistant cannula. Based on MRI coordinates, the cannula is mounted onto a stereotactic holder and guided to the targeted region of the brain, e.g. through a previously placed guide cannula. The length of each infusion cannula was measured to ensure that the distal tip extended beyond the length of the respective guide, e.g. about 1 mm, about 2 mm, about 3 mm, etc. This creates a stepped design at the tip of the cannula to maximize fluid distribution during CED procedures and minimize reflux along the cannula tract. This transition from tip to a sheath may be referred to herein as the “step”. Positioning data is optionally derived from the position of this step because of its unambiguous visibility on MRI; alternatively the tip of the cannula may be used as a reference point. It will be understood by one of skill in the art that any unambiguous marker can be utilized in positioning, and such a marker may be provided on a delivery cannula, e.g. an imaging “dot” may be integrated into the cannula design.

A delivery device may include an osmotic pump or an infusion pump. Both osmotic and infusion pumps are commercially available from a variety of suppliers, for example Alzet Corporation, Hamilton Corporation, Alza, Inc., Palo Alto, Calif.).

In one embodiment, the cannula is compatible with chronic administration. In another embodiment, the step-design cannula is compatible with acute administration.

Therapeutic agents. The methods of the invention may be applied to delivery of therapeutic agents to a targeted region of the brain. Agents of interest include, without limitation, proteins, drugs, antibodies, antibody fragments, immunotoxins, chemical compounds, protein fragments and toxins.

Examples of therapeutic agents that can be employed in the methods of this invention include GDNF family ligands, PDGF (platelet-derived growth factor) family ligands, FGF (fibroblast growth factor) family ligands, VEGF (vascular endothelial growth factor) and its homologs, HGF (hepatocyte growth factor), midkine, pleiotrophin, amphiregulin, platelet factor 4, CTGF, Interleukin 8, gamma interferon, members of the TGF-beta family, Wnt family ligands, WISP family ligands (Wnt-induced secreted proteins), thrombospondin, TRAP (thrombospondin-related anonymous protein), RANTES, properdin, F-spondin, DPP (decapentaplegic) and members of the Hedgehog family. Specific agents of interest include GDNF, neurturin, artemin, persephin, NG, BDNF, NT3, IGF-1, and sonic hedgehog. Also included are viral vectors, e.g. AAV vectors, adenovirus vectors, retrovirus vectors, etc., which are useful in the delivery of genetic constructs.

Therapeutic agents are administered at any effective concentration. An effective concentration of a therapeutic agent is one that results in decreasing or increasing a particular pharmacological effect. One skilled in the art would know how to determine effective concentration according to methods known in the art, as well as provided herein.

Dosages of the therapeutic agents and facilitating agents of this invention will depend upon the disease or condition to be treated, and the individual subject's status (e.g., species, weight, disease state, etc.) Dosages will also depend upon the agents being administered. Such dosages are known in the art or can be determined empirically. Furthermore, the dosage can be adjusted according to the typical dosage for the specific disease or condition to be treated. Often a single dose can be sufficient; however, the dose can be repeated if desirable. The dosage should not be so large as to cause adverse side effects. Generally, the dosage will vary with the age, condition, sex and extent of the disease in the patient and can be determined by one of skill in the art according to routine methods (see e.g., Remington's Pharmaceutical Sciences). The dosage can also be adjusted by the individual physician in the event of any complication.

The therapeutic agent and/or the facilitating agent of this invention can typically include an effective amount of the respective agent in combination with a pharmaceutically acceptable carrier and, in addition, may include other medicinal agents, pharmaceutical agents, carriers, adjuvants, diluents, etc. By “pharmaceutically acceptable” is meant a material that is not biologically or otherwise undesirable, i.e., the material may be administered to an individual along with the selected agent without causing any undesirable biological effects or interacting in a deleterious manner with any of the other components of the pharmaceutical composition in which it is contained.

Clinical Trials: These studies involve patients in the testing of new treatments and therapies and are part of the drug approval process. A clinical trial typically has three stages, or phases, and gauges a drug's safety, effectiveness, dosage requirements, and side effects. Patients must meet certain criteria to be enrolled in a clinical trial (which is determined for each individual study), and participation in a study is voluntary. A set of rules, or protocol, is established for each trial.

The terms “reference” and “control” are used interchangeably to refer to a known value or set of known values against which an observed value may be compared. As used herein, known means that the value represents an understood parameter, e.g., a level of expression of a cytotoxic marker gene in the absence of contact with a transfection agent.

### Methods of Use

In the methods of the invention, placement coordinates are provided for improved delivery of therapeutic agents to targeted regions of the brain. The coordinates are used with stereotactic methods to accurately position a delivery cannula. By utilizing the coordinates for cannula placement and angle of delivery, reproducible distribution of infusate in the targeted region of the brain is achieved, allowing a more effective delivery of therapeutics to the brain. Regions of interest for targeting include, without limitation, putamen, thalamus, brain stem, etc. The methods of the invention provide guidance for delivery of an agent to a “green zone”, which is a zone of the targeted region that is a suitable distance from leakage pathways of the brain.

Typically, an agent is delivered, e.g. via CED devices as follows. A catheter, cannula or other injection device is inserted into CNS tissue in the chosen subject. In view of the teachings herein, one of skill in the art could readily determine which general area of the CNS is an appropriate target. Stereotactic maps and positioning devices are available, for example from ASI Instruments, Warren, Mich. Positioning may also be conducted by using anatomical maps obtained by CT and/or MRI imaging of the subject's brain to help guide the injection device to the chosen target.

The exact position of the delivery cannula is determined using the placement guidelines of the invention. It will be understood by one of skill in the art that it is preferable to map coordinates for a targeted region experimentally on a non-human primate, and then to extrapolate from those coordinates to the desired coordinates in other primates, including humans.

Where the placement is determined experimentally, the methods set forth in the Examples may be used. An imaging agent is delivered to the targeted region of the brain, determining the distribution of the infusate; and correlating the site of cannula placement with the desired distribution, wherein the coordinates for optimal placement are those that result in appropriately contained infusate, i.e. the infusate does not spread outside of the desired target area. Regions of interest for targeting include the putamen; brain stem; cerebellum; cerebrum; corpus callosum; hypothalamus; pons; thalamus; etc.

In other embodiments, the coordinates provided herein are used to extrapolate from one species to another, through 3 dimensional modeling techniques.

The coordinate is measured relative to a reference point, for example a cannula “step”, which can be the transition point between cannula tip and sheath, a cannula tip, etc. One of skill in the art can readily extrapolate to adjust for different lengths of tip, or where the reference point is an object other than the step.

Cannula placement and definition of optimal stereotactic coordinates have important implications in ensuring effective delivery of therapeutics into the targeted brain region. Utilizing routine stereotactic localization procedures with the coordinates of the invention provide for a more effective delivery of therapeutics to the brain, and should be used in clinical therapy.

Many methods for delivering therapeutic agents to a primate brain benefit from effective localization of the agent to a region of interest. For example, leakage of growth factors away from the targeted region may have the dual disadvantage of reducing the effective amount of agent present in the targeted region, and at the same time contacting non-targeted regions with the agent. For the methods of the present invention, the targeted regions are generally homogeneous “gray matter”, consisting of neuronal cell bodies, neuropil (dendrites, axon termini, and glial cell processes), glial cells (astroglia and oligodendrocytes) and capillaries.

Gray matter comprises neural cell bodies. Gray matter is distributed at the surface of the cerebrum (i.e. cerebral cortex) and of the cerebellum (i.e. cerebellar cortex), as well as in ventral regions of the cerebrum (e.g. striatum, caudate, putamen, globus pallidus, nucleus accumbens; septal nuclei, subthalamic nucleus); regions and nuclei of the thalamus and hypothalamus; regions and nuclei of the deep cerebellum (e.g. dentate nucleus, globose nucleus, emboliform nucleus, fastigial nucleus) and brainstem (e.g. substantia nigra, red nucleus, pons, olivary nuclei, cranial nerve nuclei); and regions of the spine (e.g. anterior horn, lateral horn, posterior horn), any of which regions are suitable for targeting with the methods of the invention.

Regions that are not targeted by the methods of the invention, and which regions tend to be associated with undesirable diffusion of the infusate, are leakage pathways, including white matter. White matter mostly contains myelinated axon tracts, for example the corpus callosum (CC), anterior commissure (AC); hippocampal commissure (HC); external capsule (EC), internal capsule (IC), and cerebral peduncle (CP).

Applicants have found that containment of infusate delivered by convection enhanced delivery of agents to gray matter targeted regions requires a “green zone” relative to leakage pathways, such as the white matter or borders of the brain regions, e.g. lateral border or midline, for placement of the delivery cannula. In the methods of the invention, a delivery cannula is positioned so that the tip of the cannula is within the green zone, i.e. the zone in which infused material is contained within the targeted region.

Convection enhanced delivery (CED) infusions were retrospectively analyzed by magnetic resonance imaging (MRI) of a contrast agent for distribution in a targeted region of the brain. Infused volume (Vi) was compared to total volume of distribution (Vd), within the target region. Those infusions that provided for excellent distribution of the contrast agent were used to define an optimal target volume, or “green” zone. Those infusions that led to partial to poor distribution with leakage into adjacent anatomical structures were used to define the less desirable “blue” and “red” zones respectively. By placing the delivery cannula within the desired coordinates, quantitative containment of at least about 90% of the infusate, at least bout 95% of the infusate, at least about 98% of the infusate or more within the targeted region of the brain is achieved. These results were used to determine placement criteria that define an optimal site for infusions primate brain targeted regions.

When the delivery cannula is placed in the green zone, excellent containment of infusate within the target region may be obtained with both small volumes of less than about 30 μl volume, and large volumes of up to about 100 μl, and of volumes from about 100 μl to about 250 μl, or more. In contrast, cannula placement outside of the green zone was associated with increasing distribution of infusate as the volume of infusion grew. These data confirmed that optimal infusions could be obtained on the basis of cannula placement.

The green zone, then, is a three-dimensional mass of the targeted region, into which the tip of a delivery cannula is placed. The green zone is the inner region, surrounded by a “shell” of sufficient width to contain infusate.

In general, the “green zone” for positioning of the delivery cannula tip is sufficiently within a targeted gray matter region to avoid leakage pathways.

For example, where the targeted region is within the cerebrum, e.g. the cerebral cortex, the striatum, the putamen, caudate, etc. the placement coordinates may be mapped relative to axon tracts such as the corpus callosum (CC), anterior commissure (AC); external capsule (EC), and internal capsule (IC), where the green zone is a distance of at least about 2 mm, at least about 2.5 mm, usually at least about 3 mm, and in target regions of sufficient size, the green zone may be at least about 3.5 mm, at least about 4 mm; each distance being measured from the axon tracts, e.g. white matter, as shown in Example 1.

Where the targeted region is the thalamus or hypothalamus, the “green zone” is defined by the borders of the targeted region, and are, for example at least 2.5 mm, at least 2.8 mm, at least 3.0 mm to entry point; at least 1.8, at least 2.0, at least 2.2 mm from the lateral border; and at least 4.5 mm, at least 4.75, at least 5 mm from midline, as shown in Example 2.

Where the targeted region is within the brainstem, e.g. substantia nigra, red nucleus, pons, olivary nuclei, cranial nerve nuclei, etc., the “green zone” is defined by the borders of the targeted region, for example as at least 2.8 mm, at least 3.0 mm, at least 3.5 mm to entry point; at least 2.5, at least 2.75, at least 2.92 mm from the lateral border of brainstem; and at least 1.25 mm, at least 1.5, at least 1.6 mm from midline, as shown in Example 2.

Desirably the length of the cannula tip is at least about 1 mm, at least about 1.5 mm, at least about 2 mm, at least about 2.5 mm, at about 3 mm, at least about 3.5 mm, at least about 4 mm at least about 4.5 mm, at least about 5 mm or more.

By placing the delivery cannula at the coordinate designated above, quantitative containment of at least about 90% of the infusate, at least about 95% of the infusate, at least about 98% of the infusate or more within the targeted region of the brain is achieved.

In some embodiments of the invention, a system is provided for accurate placement of a drug delivery cannula to a targeted region of the brain. Such systems comprise the coordinate information as set forth herein, in a stereotactic delivery system. Such systems may further comprise one or more of a delivery cannula; pump; and therapeutic agent.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### EXPERIMENTAL

### Example 1

### Optimal Region of the Putamen for Image-Guided Convection-Enhanced Delivery of Therapeutics in Human and Non-Human Primates

### Materials and Methods

Experimental subjects and study design. Thirteen normal adult NHP, including 11 Rhesus macaques (7 male and 4 female, aged from 8 to 18 years; mean age 11.9 years, weight 4-9.4 kg) and 2 Cynomolgus monkeys (one male and one female, age 7 years for both; weight 5 and 7 kg respectively) were the subjects in the present study. Experimentation was performed according to the National Institutes of Health guidelines and to the protocols approved by the Institutional Animal Care and Use Committee at the University of California San Francisco (San Francisco, Calif.) and at Valley Biosystems (Sacramento, Calif.). Thirteen animals received a total of 25 intracranial infusion of GDL (2 mM) or free Gadoteridol (2 mM, Prohance; Bracco Diagnostics, Princeton, N.J.) into the putamen. Infusions were performed by previously established CED techniques for NHP (Bankiewicz, Eberling et al. 2000). GDL were prepared as previously described (Fiandaca, Varenika et al. 2008) (Krauze, McKnight et al. 2005).

Infusion procedure. Primates received a baseline MRI before surgery to visualize anatomical landmarks and to generate stereotactic coordinates of the proposed target infusion sites for each animals. NHPs underwent neurosurgical procedures to position the MRI-compatible guide cannula over the putamen. Each customized guide cannula was cut to a specified length, stereotactically guided to its target through a burr-hole created in the skull, and secured to the skull by dental acrylic. The tops of the guide cannula assemblies were capped with stylet screws for simple access during the infusion procedure. Animals recovered for at least 2 weeks before initiation of infusion procedures. Animals were anesthetized with isoflurane (Aerrane; Ohmeda Pharmaceutical Products Division, Liberty Corner, N.J.) during real-time MRI acquisition. Each animal's head was placed in an MRI-compatible stereotactic frame, and a baseline MRI was performed. Vital signs, such as heart rate and PO₂, were monitored throughout the procedure.

Briefly, the infusion system consisted of a fused silica reflux-resistant cannula (Fiandaca, Varenika et al. 2008) (Krauze, McKnight et al. 2005) that was connected to a loading line (containing GDL or free Gadoteridol), an infusion line with oil, and another infusion line with trypan blue solution. A 1-ml syringe (filled trypan blue solution) mounted onto a micro-infusion pump (BeeHive, Bioanalytical System, West Lafayette, Ind.), regulated the flow of fluid through the system. Based on MRI coordinates, the cannula was mounted onto a stereotactic holder and manually guided to the targeted region of the brain through the previously placed guide cannula. The length of each infusion cannula was measured to ensure that the distal tip extended 3 mm beyond the length of the respective guide. This created a stepped design at the tip of the cannula to maximize fluid distribution during CED procedures and minimize reflux along the cannula tract. We refer to this transition from fused silica tip to a fused silica sheath as the “step”, and all positioning data is derived from the position of this step because of its unambiguous visibility on MRI.

After securing placement of the infusion cannula, the CED procedures were initiated with real-time MRI data being acquired (real-time convective delivery, RCD). We used the same infusion parameters for every NHP infused throughout the study. Infusion rates were as follows: 0.1 μl/min was applied when lowering cannula to targeted area and increased at 10-min intervals to 0.2, 0.5, 0.8, 1.0, and 2.0 μl/min. Approximately 15 min after infusion, the cannula was withdrawn from the brain. Four animals received multiple infusions. Each animal had at least a 4-week interval between each infusion procedure.

Magnetic resonance image (MRI). NHPs were sedated with a mixture of ketamine (Ketaset, 7 mg/kg, IM) and xylazine (Rompun, 3 mg/kg, IM). After sedation, each animal was placed in a MRI-compatible stereotactic frame. The ear-bar and eye-bar measurements were recorded, and an intravenous line was established. MRI data was then obtained, after which animals were allowed to recover under close observation until able to right themselves in their home cages. MR images of brain in 9 NHP were acquired on a 1.5T Siemens Magnetom Avanto (Siemens AG, Munich, Germany). Three-dimensional rapid gradient echo (MPRAGE) images were obtained with repetition time (TR)=2110 ms, echo time (TE)=3.6 ms, and a flip angle of 15°, number of excitations (NEX)=1 (repeated 3 times), matrix=240×240, field of view (FOV)=240×240×240, and slice thickness=1 mm. These parameters resulted in a 1-mm₃ voxel volume. The scanning time was approximately 9 min. MR images in 4 NHP were acquired on a 1.5-T Sigma LX scanner (GE Medical Systems, Waukesha, Wis.) with a 5-inch surface coil on the subject's head, parallel to the floor. Spoiled gradient echo (SPGR) images were T1-weighted and obtained with a spoiled grass sequence, a TR=2170 ms, a TE=3.8 ms, and a flip angle of 15°. The NEX=4, matrix=256×192, FOV=16 cm×12 cm, slice thickness=1 mm. These parameters resulted in a 0.391 mm₃ voxel volume. Scanning time was approximately 11 min.

MR images in 4 NHP were acquired on a 1.5-T Sigma LX scanner (GE Medical Systems, Waukesha, Wis.) with a 5-inch surface coil on the subject's head, parallel to the floor. Spoiled gradient echo (SPGR) images were T1-weighted and obtained with a spoiled grass sequence, a TR=2170 ms, a TE=3.8 ms, and a flip angle of 15°. The NEX=4, matrix=256×192, FOV=16 cm×12 cm, slice thickness=1 mm. These parameters resulted in a 0.391 mm₃ voxel volume. Scanning time was approximately 11 min.

Volume and distance measurements in NHP brain. MR images were obtained from each real-time convective delivery (RCD), and used to measure distance from cannula step to corpus callosum (CC), internal capsule (IC) and external capsule (EC). The measurements were made on an Apple Macintosh G4 computer with OsiriX® Medical Image Software (v2.5.1). OsiriX software reads all data specifications from DICOM (digital imaging and communications in medicine) formatted MR images obtained via local picture archiving and communication system (PACS). The distances from cannula step to each above-mentioned structure were manually defined, and then calculated by the software. All the distances were measured in the same manner on MRI sections.

The X, Y and Z axial values of cannula step location in green zone were determined with 2D orthogonal MR images generated by OsiriX software, where MR images were projected in all three dimensions (axial, coronal and sagittal). We used midpoint of the anterior commissure-posterior commissure (AC-PC) line as zero point (0,0,0) of three-dimensional (3D) brain space. Briefly, AC-PC line was drawn on midsagittal plane of MRI, and the midpoint of AC-PC line was determined. The horizontal and vertical plane through the midpoint of AC-PC line was then obtained, and they could be shown on all the three plans simultaneously. The X, Y and Z axial values of cannula step were then obtained by measurements of distance from cannula step to midline on coronal MRI plane (X value), distance anterior (or posterior) to the midpoint of AC-PC line of the coronal MRI plane (Y value), and the distance above (or below) axial plane incorporating the AC-PC line on MRI (Z value). All the distances were measured (in millimeters) in the same manner on MRI sections for each case.

MR images were also used for volumetric quantification of distribution of Gadoteridol. The Vd of Gadoteridol in the brain of each subject was also quantified on an Apple Macintosh G4 computer. ROI derived in the putamen and white matter track were manually defined, and software then calculated the area from each MR image, and established the volume of the ROI, based on area defined multiplied by slice thickness (PACS volume). The boundaries of each distribution were defined in the same manner in the series of MRI sections. The sum of the PACS ROI volumes (number of MRI slices evaluated) for the particular distribution being analyzed determined the measured structure volume. The defined ROI volumes allowed for 3D image reconstruction with BrainLAB software (BrainLAB, Heimstetten, Germany). MRIs were evaluated and all measurements performed by two independent observers blind to each other. In a preliminary comparison of distances measured by the two observers in NHPs, there was no significant difference between the mean values obtained.

Statistical Analysis. The distance from cannula step to corpus callosum, internal capsule and external capsule obtained when the step was located in different zones were compared across subject groups by Student's t-test. The criterion for statistical significance for all tests was p<0.05.

### Results

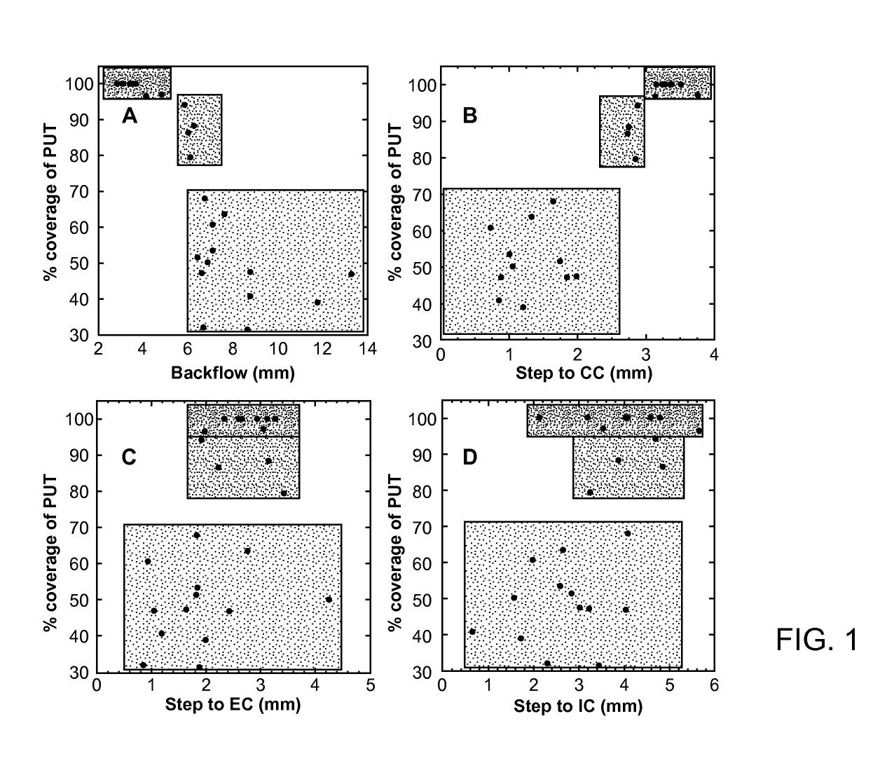

In this study, thirteen NHP received twenty-five putaminal infusions. Real-time MR images of NHP brain were obtained from each RCD to evaluate the distribution of Gadoteridol, and to measure the distance from step of cannula in the putamen to CC, IC and EC based on the location of the cannula step. We observed that some infusions resulted in poor containment of tracer within putamen with significant distribution into adjacent white matter tracts (WMT) of the corpus callosum (CC) and occasionally internal (IC) and external (EC) capsules, whereas others distributed tracer only into putamen (Table 1). If the percent of infused tracer contained within the putamen is plotted against each variable (FIG. 1), it is apparent that reflux along the cannula correlates (FIG. 1A) with a sharp decline in distribution of infusate into the putamen (PUT). Containment of tracer within putamen (PUT) in excess of 95% is achievable with backflows of less than about 5 mm. The tip length in these experiments was 3 mm. Subsequent correlations between PUT coverage and anatomical coordinates revealed also that another key variable appears to be the distance from the corpus callosum (CC) to the cannula step (FIG. 1B). In 8 infusions in which putaminal containment exceeded 95%, the cannula step-to-CC ranged from 3.14 mm to 3.76 mm with mean distance of 3.35±0.08 mm, the step-to-IC ranged from 2.13 mm to 5.65 mm with mean distance of 4.01±0.42 mm, and the step-EC ranged from 1.98 mm to 3.28 mm with mean distance of 2.75±0.17 mm.

We conclude that the step-to-CC distance should exceed about 3 mm for optimal containment of infusate within putamen. The distance from the cannula step to IC and EC (FIG. 1 C, D) correlated poorly with putaminal containment. We defined the spatial limits associated with essentially quantitative putaminal containment of tracer as the “green zone”. A corresponding “blue zone”, associated with putaminal containment of tracer in from 79% to 94% with mean of 87%±3% indicative of a small amount of leakage into the CC, was also defined in 4 cases. Here the step-to-CC ranged between 2.74 mm and 2.88 mm with mean distance of 2.81±0.04 mm; the step-IC ranged from 3.26 mm to 4.86 mm with mean distance of 4.18±0.37 mm, and the step-EC from 1.92 mm to 3.43 mm with mean distance of 2.68±0.36 mm.

Similarly, a “red zone” was defined in 13 cases where tracer was poorly confined to PUT, ranging from 31% to 67% of PUT with a mean of 49%±0.05%, indicating a large amount of leakage into the CC, EC and IC. In these infusions, the step-to-CC ranged from 0.12 mm to 1.99 mm with mean distance of 1.26±0.16 mm; the step-to-IC ranged from 0.65 mm to 4.08 mm with mean distance of 2.63±0.27 mm, and the step-to-EC from 0.85 mm to 4.25 mm with mean distance of 1.88±0.25 mm.

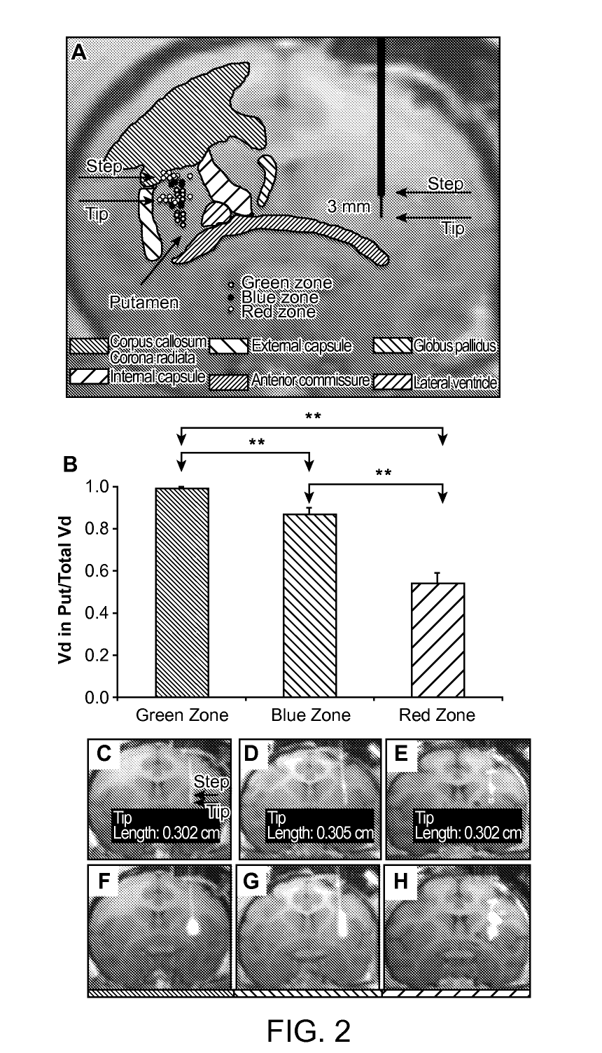

Volume of distribution of Gadoteridol in the brain. When the step was placed in the “green zone” in 8 cases, excellent Vd of Gadoteridol was obtained in the putamen, ranging from 52.9 to 174.1 mm³ with mean volume of 116.4±0.04 mm³ (FIGS. 2A and 2B). Two cases were found to have minor leakage of Gadoteridol into CC at the end of infusion, and their Vd in white matter tract (WMT) was 2.7 and 6.1 mm³, respectively. Representative MRI are shown in FIGS. 2C and 2F.

In 4 cases in which the step was placed in the blue zone, the Vd of Gadoteridol in the putamen ranged from 40.7 to 261.9 mm³ with mean volume of 139.6±0.05 mm³ (FIGS. 2A and 2B). All 4 cases were found to have leakage into CC. When leakage was first seen, the infusion volume ranged from 4.7 to 10.5 μl with mean volume of 6.9±0.9 μl. The final Vd in WMT ranged from 6.3 to 40.7 mm₃ with mean volume of 19.4±0.01 mm₃. Representative MRI is shown in FIGS. 2D and 2G.

Placement of the step in the “red zone” in 13 cases produced a Vd of Gadoteridol from 17.7 to 97.5 mm₃ with mean volume of 62.1±0.01 mm₃ (FIGS. 2A and 2B). All 13 cases were found to have considerable leakage into CC with variable leakage into IC and EC. When leakage was first seen, the infusion volume was between 1.6 and 21.8 μl with mean volume of 7.9±1.7 μl. The final Vd in WMT ranged from 26.7 to 152.2 mm³ with a mean volume of 66.8±0.01 mm³. Of 17 cases with relatively large leakage during CED, leakage into CC was found in all 17 cases (100%), into IC in 3 cases (17.6%) and into EC in one case (5.9%). Representative MRI is shown in FIGS. 2E and 2H.

Coordinates for green zone in the putamen of 3D brain space in NHP. The midpoint of the AC-PC line was defined as the zero point (0,0,0) of a 3D brain space. Based on the coordinate calculations for the cannula step by MRI, the target for green zone in the putamen ranged from 9.57 to 14.95 mm with mean distance of 11.85±0.56 mm lateral (X coordinate), from 5.88 to 8.93 mm with mean distance of 7.36±0.49 mm anterior to the of AC-PC midpoint (Y coordinate), and from 1.64 to 4.47 mm with mean distance of 3.62±0.40 mm superior to the AC-PC axial plane (Z coordinate).

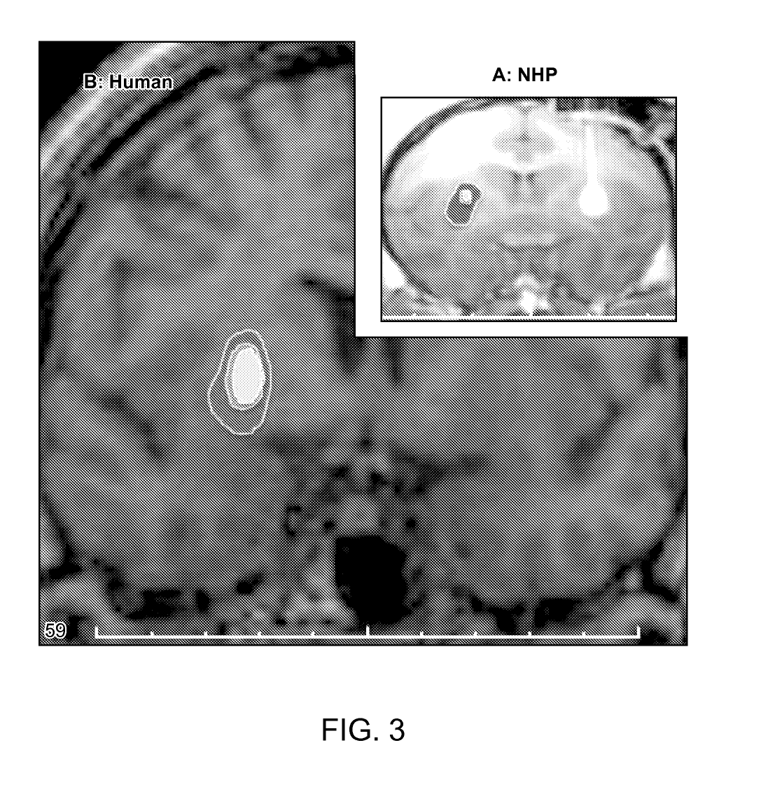

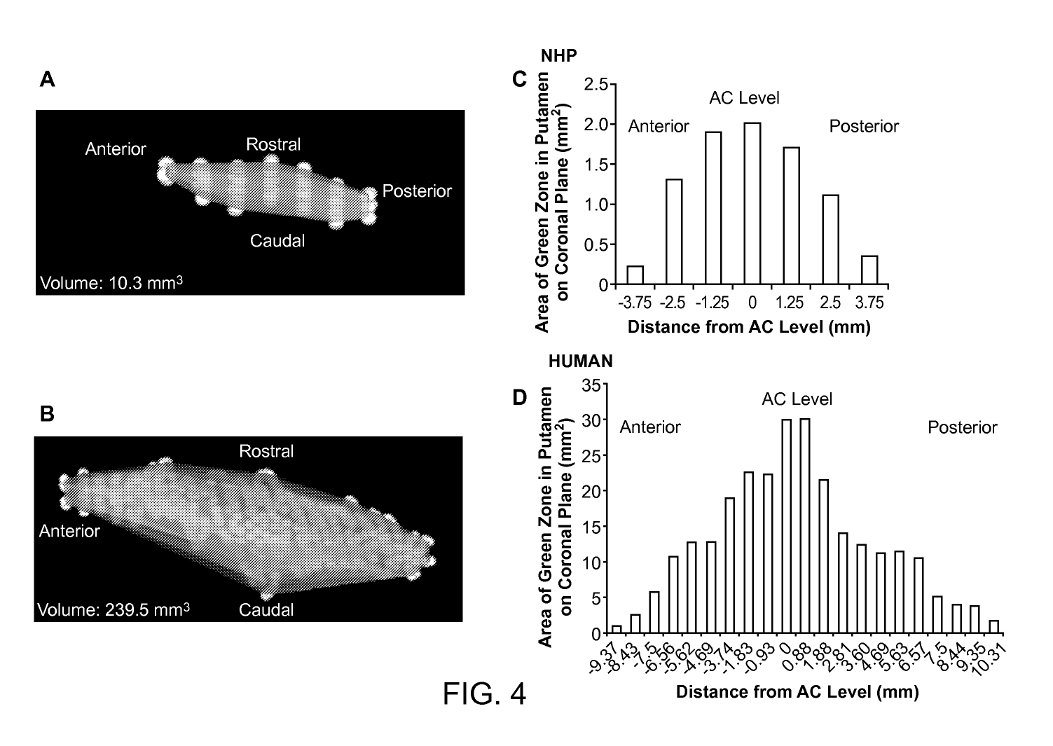

RGB zones for cannula step in the putamen of NHP. On the basis of these analyses, we have defined coordinates for putaminal infusions that identify preferred cannula characteristics and optimal distances from major structures in the brain (RBG zones). The “green zone” is defined as a volume at least 3 mm ventral to the CC, at least 6 mm away from the AC (3 mm from cannula tip to AC plus 3 mm of tip length) vertically, greater than 2.75 mm from EC laterally, and more than 3 mm from IC medially. If globus pallidus is included, then the optimal distance from IC is more than 4.01 mm. The “blue zone” is defined as a thick shell surrounding the “green zone” of which the outer border of “blue zone” is approximately 0.5 mm from the outer edge of the green zone. Finally, the “red zone” is defined as the area from the outer border of the blue zone to the margin of the putamen. Based on these parameters, RBG zones for cannula placement in the NHP putamen were defined on MRI (FIG. 3A). Next, we also outlined “green zone” only, and then calculated the volume of the green zone to be 10.3 mm³ with an anterior-posterior length of 8.5 mm (FIG. 4A).

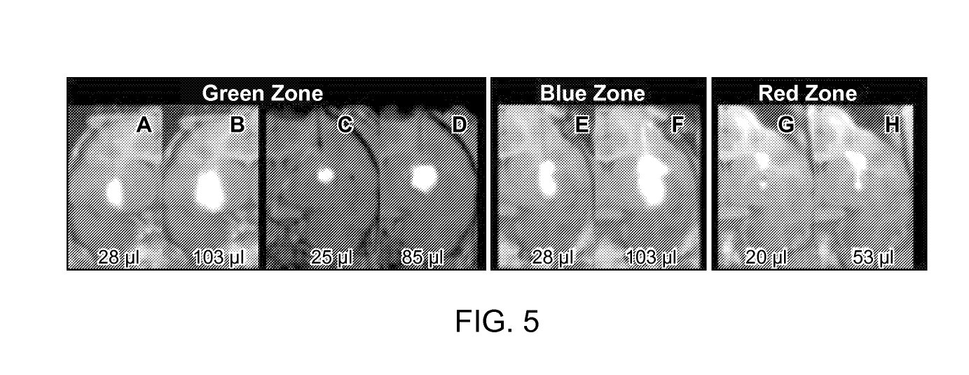

Containment vs. distribution in NHP putamen. In the above studies, only small amounts (<30 μl) of tracer were infused sufficient to register the relative partitioning of infusate into PUT, CC, IC, and/or EC. We wished, however, to show that infusion of larger volumes into green zone would faithfully distribute into PUT with no untoward non-putaminal distribution. By retrospective examination of other putaminal infusions in NHP, we found that in animals where cannula placement was in the green zone, excellent containment of infusate within PUT was seen at small (<30 μl) and large (>100 μl) volumes (FIG. 5). In contrast, cannula placement in blue zone was associated with increasing distribution of infusate into WMT as the volume of infusion grew. These representative data confirmed that, with a defined RBG zone system in hand, we could identify optimal infusions on the basis of optimal cannula placement alone.

RBG zones in the putamen of human brain. We used the parameters for RBG zone obtained from NHP to predict RBG zones in the putamen of human brain (FIG. 3B, FIG. 4), which serve as a guide to RBG zones in human PUT when local therapies such as gene transfer or protein administration are translated into clinical therapy. We also outlined the green zone on serial MR images and then calculated the area from each MR image to predict that the volume of the green zone is 239.5 mm₃ with an anterior-posterior distance of 19.7 mm. The RBG zones for cannula step in the PUT of NHP and human are also compared as shown in FIG. 3 on the same scale.

In the present study, we correlated the precise stereotactic placement of the infusion cannula in PUT of NHPs with the efficiency of MRI tracer distribution into the PUT. Clearly, some infusions were associated with excellent containment of tracer, others were somewhat less efficient and displayed some evidence of reflux. A number of infusions, however, were poorly contained within PUT and were associated with leakage of tracer primarily into corpus callosum WMT. Analysis of these data (FIG. 1) indicated that the variables most determinant of putaminal containment were the length of the cannula tip and the distance of the cannula step to the corpus callosum. Distance of the step to the internal and external capsules correlated poorly with containment. The correlation between stereotactic coordinates of the cannula and resulting PUT:WMT partition of tracer permitted us to define a putaminal “green zone”, a 3D space in which cannula placement is optimal and convection of infusate into putamen is optimal. Similarly, a “blue zone” was defined as sub-optimal but still acceptable in some cases, and a “red zone” associated with unacceptable results. In addition, we showed that the “green zone” predicts effective Vd into PUT where untoward leakage of infusate into WMT may be avoided.

Reflux up the cannula track cause a disruption of the pressure gradient which compromises distribution of the infusate in the PUT, leading to reduced Vd. Leak of the infusate into the CC is most common and it depends on proximity of the step to CC, as we show in this report. If the step is close to CC, combined with the fact that the cannula axis runs through it, reflux will always occurs in the direction of the cannula axis.

We used the NHP “green zone” to predict a corresponding zone in human PUT. Our computational analysis has shown that humans have a proportionately larger green zone compared with NHP, and that the 23-fold difference in volume of green zone is due to the size difference between NHP and human PUT as shown previously (Yin et al. 2009 J Neurosci Methods 176(2): 200-5). Apart from the obvious difference in size, the overall morphology of the green zone is remarkably similar. This knowledge is critical in obtaining excellent Vd of therapeutics in the putamen of patients without significant leakage into surrounding anatomical structures.

With the more widespread use of CED in the treatment of human neurological diseases, as has been previously described (Eberling et al 2008 Neurology 70(21):1980-3), controlled distribution of therapeutic agents within brain structures is essential for any approach utilizing gene or molecular therapy. It is important for optimizing efficacy to cover the entire targeted treatment volume while avoiding adjacent regions of the brain or CSF pathways. It has been very difficult to predict the distribution of therapeutics delivered by CED, due to a lack of understanding of optimal cannula placement under these circumstances. This is true for delivery of chemotherapeutic agents to brain tumors, and for infusion of growth factors, enzymes, and viral vectors in PD patients.

Emergence of iMRI technology for intraoperative imaging of functional neurosurgical therapeutic interventions, such as MRI-guided placement of DBS stimulating electrodes in PD (Larson et al. 2008 Stereotact Funct Neurosurg 86(2): 92-100; Martin et al. 2009 Top Magn Reson Imaging 19(4): 213-21), is another example of image-guided therapy application in the brain. Precise targeting of “green zone” for CED can be accomplished by use of skull mounted aiming devices and the iMRI unit. In addition to visualization of accurate placement of the infusion cannula, desired distribution of the therapeutic agent can be achieved by visualization of the CED and subsequent control of the infusion procedure.

In summary, the present study provides the first quantitative analysis by MRI of cannula placement and distribution of Gadoteridol, and introduces a definition of RBG zones in the NHP putamen. Moreover, real-time visualization of cannula placement by MRI, and subsequent precise control of the extent of Gadoteridol distribution, addresses an important safety issue, especially when parenchymal infusion of large volumes is necessary and leakage or excessive distribution may be undesirable. Cannula placements in the RBG zones developed from our translational non-human primate studies have significant implications for clinical trials featuring CED of various therapeutic agents into the putamen for PD. Similar RBG zones can be defined for other brain regions as well, such as thalamus and brainstem, thereby establishing reliable coordinates for neurosurgical infusions of therapeutic agents in the clinic.

TABLE 1
Measurement of distance from step to CC, IC and EC, length of backflow
and percent of distribution of MRI tracer in the putamen.

| | | | | | | Vd in |
|---|---|---|---|---|---|---|
| | | | | | | put/Vd |
| | Step to | Step to | Step to | Reflux | % of | of |
| Infusion | CC (mm) | IC (mm) | EC (mm) | (mm) | PUT | leakage |
| 1 | 3.38 | 4.8 | 2.94 | 3.54 | 100% | ND |
| 2 | 3.24 | 4.04 | 3.28 | 3.1 | 100% | ND |
| 3 | 3.76 | 3.54 | 3.06 | 4.83 | 97.1% | ND |
| 4 | 3.14 | 5.65 | 1.98 | 4.14 | 96.6% | ND |
| 5 | 3.36 | 4.1 | 2.66 | 3.42 | 100% | ND |
| 6 | 3.51 | 4.6 | 2.34 | 3.68 | 100% | ND |
| 7 | 3.15 | 2.13 | 2.61 | 2.84 | 100% | ND |
| 8 | 3.28 | 3.2 | 3.13 | 3.39 | 100% | ND |
| 9 | 2.88 | 4.7 | 1.92 | 5.85 | 94.2% | 16.30 |
| 10 | 2.85 | 3.26 | 3.43 | 6.1 | 79.5% | 3.88 |
| 11 | 2.74 | 4.86 | 2.23 | 5.99 | 86.5% | 6.43 |
| 12 | 2.75 | 3.88 | 3.15 | 6.26 | 88.4% | 7.62 |
| 13 | 1.65 | 4.08 | 1.83 | 6.74 | 67.9% | 2.11 |
| 14 | 1.01 | 2.59 | 1.84 | 7.08 | 53.5% | 1.15 |
| 15 | 1.75 | 2.84 | 1.82 | 6.43 | 51.5% | 1.07 |
| 16 | 1.85 | 4.04 | 2.43 | 13.29 | 47.0% | 0.89 |
| 17 | 1.96 | 3.45 | 1.88 | 8.65 | 31.4% | 0.46 |
| 18 | 0.12 | 2.31 | 0.85 | 6.66 | 32.1% | 0.47 |
| 19 | 0.86 | 0.65 | 1.19 | 8.76 | 40.8% | 0.69 |
| 20 | 0.73 | 1.99 | 0.94 | 7.09 | 60.7% | 1.54 |
| 21 | 1.33 | 2.65 | 2.76 | 7.61 | 63.6% | 1.75 |
| 22 | 1.99 | 3.03 | 1.64 | 8.78 | 47.5% | 0.91 |
| 23 | 1.21 | 1.73 | 1.99 | 11.78 | 39.0% | 0.64 |
| 24 | 0.89 | 3.23 | 1.05 | 6.62 | 47.2% | 0.89 |
| 25 | 1.05 | 1.57 | 4.25 | 6.88 | 50.2% | 1.01 |

Spatial coordinates correlated with length of backflow and percent of containment of tracer within the putamen. The ratio of Vd in PUT to Vd of leakage was obtained by dividing the volume of distribution of tracer in the putamen by the volume of leakage of tracer into white matter tract. CC, corpus callosum; IC, internal capsule; EC, external capsule; PUT, putamen; and Vd, volume of distribution.

### Example 2

### Real-Time Visualization and Characterization of Gadoteridol Delivery into Thalamus and Brain Stem in Non-Human Primates by Magnetic Resonance Imaging

In this study, six NHP received 22 infusions into thalamus and brainstem. Real-time MR images of NHP brain were obtained from each RCD to evaluate the distribution of Gd and to measure the distance from cannula step in the thalamus or brainstem to midline, lateral border and cannula entry point to targeted structure, respectively, based on the location of the cannula step.

### Experimental Subjects and Study Design

Six normal adult NHP, including 4 Cynomolgus monkeys (2 male and 2 female, age from 7 to 8 years; mean age 8.2 years, weight 5-12.8 kg) and 2 Rhesus macaques (1 male, age 10 years, weight 12.2 kg; 1 female, age 8 years, weight 6 kg) were enrolled in the study. Experiments were performed according to the National Institutes of Health guidelines under protocols approved by the Institutional Animal Care and Use Committee at the University of California San Francisco (San Francisco, Calif.) and at Valley Biosystems (Sacramento, Calif.). These animals received a total of 22 intracranial infusions of gadoteridol (Gd, 2 mM) into the thalamus and brainstem. Infusions were performed by previously established CED techniques for NHP.

Infusion procedure. primates received a baseline MRI prior to surgery to visualize anatomical landmarks and to generate stereotactic coordinates of the proposed infusion target sites. NHP underwent stereotactic placement of the MRI-compatible plastic guide cannula array (12 mm diameter×14 mm height containing 27 access holes) for CED into the thalamus and brainstem. Each guide cannula array was secured to the skull with plastic screws and dental acrylic. After placement of the guide cannula array, animals recovered for at least 2 weeks before initiation of infusion procedures. On the day of infusion, animals were anesthetized with isoflurane (Aerrane; Ohmeda Pharmaceutical Products Division, Liberty Corner, N.J.). Each animal's head was then placed in an MRI-compatible stereotactic frame, and a baseline MRI was performed. Vital signs, such as pulse and PO₂, were monitored throughout the procedure. Briefly, the infusion system consisted of a fused silica reflux-resistant cannula that was connected to a loading line (containing Gd), an infusion line with oil, and another infusion line with trypan blue solution. A 1-ml syringe (filled trypan blue solution) mounted onto a Harvard MRI-compatible infusion pump (Harvard Bioscience Company, Holliston, Mass.), regulated the flow of fluid through the delivery cannula. Based on MRI coordinates, the cannula was inserted into the targeted region of the brain through the previously placed guide cannula array.

The length of each infusion cannula was measured to ensure that the distal tip extended 3 mm beyond the cannula step. This created a stepped design that was proximal to the tip of the cannula, maximizing fluid convection during CED while minimizing reflux along the cannula tract. In the text, we refer to this transition from fused silica tip to a fused silica sheath as the “step”, and all positioning data is derived from the position of this step due to its unambiguous visibility on MRI. We maintained positive pressure in the infusion cannula during its insertion into the brain to minimize possible tip occlusion during cannula insertion. After securing placement of the infusion cannula, the CED procedures were initiated acquisition of MRI data in real time (real-time convective delivery, RCD). We used the same infusion parameters for every NHP infused throughout the study. Infusion rates were as follows: 0.1 μl/min was applied when lowering cannula to targeted area (to prevent tissue from entering the tip) and, upon achieving the target, increased at 10-min intervals to 0.2, 0.5, 0.8, 1.0, and 2.0 μl/min. Approximately 15 min after infusion, the cannula was withdrawn from the brain. Four animals received multiple infusions. Each animal had at least a 4-week interval between each infusion procedure.

Magnetic resonance image (MRI). NHP were sedated with a mixture of ketamine (Ketaset, 7 mg/kg, IM) and xylazine (Rompun, 3 mg/kg, IM). After sedation, each animal was placed in a MRI-compatible stereotactic frame. The ear-bar and eye-bar measurements were recorded, and an intravenous line was established. MRI data was then obtained, after which animals were allowed to recover under close observation until able to right themselves in their home cages. MR images of brain in 14 CED in 4 NHP were acquired on a 1.5T Siemens Magnetom Avanto (Siemens AG, Munich, Germany). Three-dimensional (3D) rapid gradient echo (MP-RAGE) images were obtained with repetition time (TR)=2110 ms, echo time (TE)=3.6 ms, and a flip angle of 15°, number of excitations (NEX)=1 (repeated 3 times), matrix=240×240, field of view (FOV)=240×240×240, and slice thickness=1 mm. These parameters resulted in a 1-mm³ voxel volume. The scanning time was approximately 9 min.

MR images of 8 CED in 2 NHP were acquired on a 1.5-T Sigma LX scanner (GE Medical Systems, Waukesha, Wis.) with a 5-inch surface coil on the subject's head, parallel to the floor. Spoiled gradient echo (SPGR) images were T1-weighted and obtained with a spoiled grass sequence, a TR=2170 ms, a TE=3.8 ms, and a flip angle of 15°. The NEX=4, matrix=256×192, FOV=16 cm×12 cm, slice thickness=1 mm. These parameters resulted in a 0.391 mm³ voxel volume. Scanning time was approximately 11 min.

Volume and distance measurements in NHP brain. MR images, obtained from each RCD, were used to measure the distance from the cannula step to the midline (step-midline), to cannula entry point (step-entry) to the target region (thalamus or brainstem), and to the lateral borders (step-lateral), of the target regions. The measurements were made on an Apple Macintosh G4 computer with OsiriX® Medical Image Software (v2.5.1). OsiriX software reads all data specifications from DICOM (digital imaging and communications in medicine) formatted MR images obtained via a local picture archiving and communication system (PACS). The distances from the cannula step to each of the above-mentioned points were manually defined, and then calculated by the software after each point was selected. All distances were measured in the same manner on all MRI sections.

The X, Y and Z coordinate values of each cannula step location in the green zone were determined with 2D orthogonal MR images generated by OsiriX software, where MR images were projected in all three planes (axial, coronal and sagittal). We used the midpoint of the anterior commissure-posterior commissure (AC-PC) line, midcommissural point (MCP), as the zero point (0,0,0) in three-dimensional (3D) brain space. Briefly, the AC-PC line was drawn on the mid-sagittal plane, and the MCP was defined. Orthogonal horizontal (axial) and vertical (coronal) planes through the MCP were then determined, with the axial plane containing the AC-PC line, along with the mid-sagittal plane. The X, Y and Z values of the cannula step were then obtained by measurements of the distance from cannula step to midline on the coronal MRI plane (X value), the distance anterior (or posterior) to the MCP on the axial MRI plane (Y value), and the distance above (or below) the AC-PC line on the sagittal MRI (Z value). All the distances were measured (in millimeters) in the same manner on MRI sections for each case.

MR images were also used for volumetric quantification (Vd) of the distribution of Gd. The Vd of Gd in the brain of each subject was also quantified on an Apple Macintosh G4 computer. Regions of interest (ROI) were manually defined by outlining the enhancing area of infusion in the thalamus or brainstem, and in surrounding structures. The Osirix software then calculated the area from each MR image, and established the volume of the ROI, based on the areas defined multiplied by slice thickness (PACS volume). The boundaries of each distribution were defined in the same manner in the series of MRI sections. The sum of the PACS ROI volumes (number of MRI slices evaluated) for the particular distribution being analyzed determined the measured volume. The defined ROI volumes allowed for 3D image reconstruction with BrainLAB software (BrainLAB, Heimstetten, Germany).

Statistical Analysis. The distribution of Gd and the distance variables (cannula step to midline; cannula step to region entry point; cannula step to lateral border of each region) were compared across subject groups by Student's t-test. The criterion for statistical significance for all tests was p<0.05.

### Results

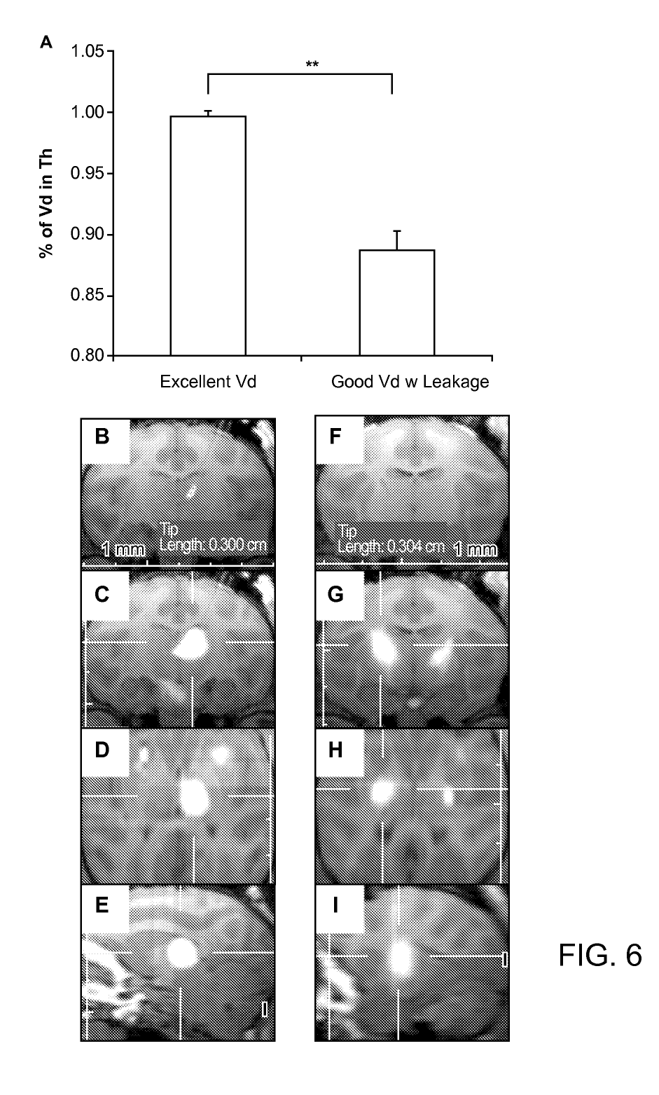

Distribution of Gadoteridol in the thalamus during CED. Of 14 infusions performed in the thalamus, excellent distribution of Gd was achieved in 8 cases (57.1%), and their Vd ranged from 159.1 to 660.3 mm³ with mean volume of 405.6±66.6 mm³. FIG. 6 shows the percent of Vd of Gd in the thalamus vs total Vd in thalamus and WMT, which was 100% in all 8 cases, indicating no leakage of Gd into the WMT.

In 6 cases (42.9%), good distribution of Gd in the thalamus was obtained with leakage into WMT in 5 cases and into lenticular fasciculus (Lenf) in 4 cases. The Vd of Gd in the thalamus ranged from 58.5 to 267.6 mm³ with mean volume of 191.3±38.1 mm³. The percent of Vd in the thalamus ranged from 86.0% to 93.1% with mean of 89.0%±1.3% (FIG. 6), which indicate some leakage into the surrounding structures. The Vd of leakage ranged from 8.3 to 43.7 mm³ with mean volume of 24.3±7.0 mm³. There was significant difference in the distributions of Gd in the thalamus between excellent Vd and good Vd with leakage. Representative MRIs show cannula step placement (FIGS. 6B and 6F) and distribution of Gd (FIG. 6C to 6E and 6G to 6I) in the thalamus.

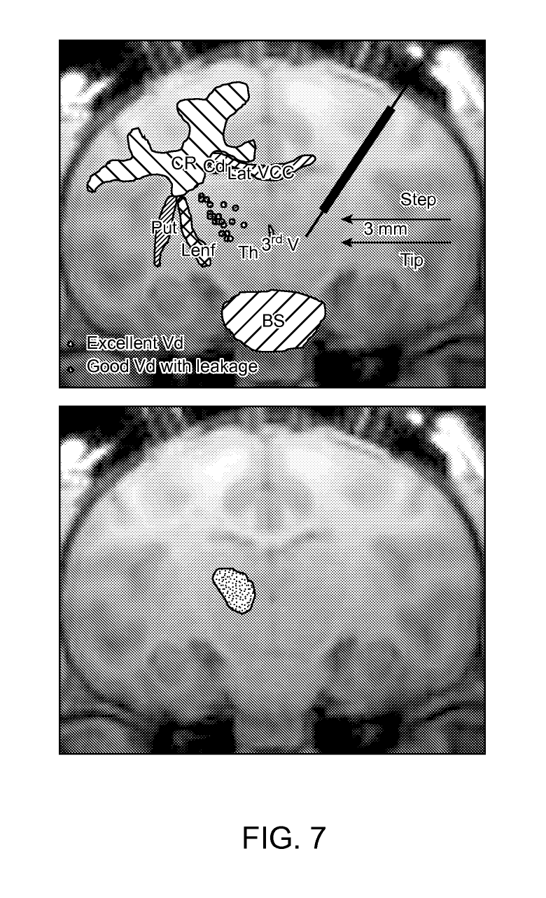

Measurements of parameters for cannula step placement in the thalamus. We observed that some infusions resulted in good containment of tracer within thalamus with some distribution into adjacent WMT and Lenf, whereas others distributed tracer only into thalamus. During CED, the Vd for a given agent depends on many factors. In our experience, the important component of successful CED is likely to be cannula placement. Therefore, MR images were used to measure distance from cannula step to midline (step-to-mid), lateral border (step-to-lat), and cannula entry point (step-to-ent) of thalamus. Cannula placement in the thalamus is shown in FIG. 7.

In 7 cases with excellent containment of Gd in the thalamus, the step-to-mid ranged from 4.99 mm to 7.73 mm with mean distance of 6.24±0.36 mm, the step-to-ent ranged from 2.82 mm to 4.59 mm with mean distance of 3.96±0.29 mm, and the step-to-lat ranged from 2.16 mm to 6.95 mm with mean distance of 3.58±0.63 mm. The angle between cannula and horizontal line ranged from 58.85 to 66.67 degree with a mean 63.90±1.02 degree.

In 5 cases with good containment of Gd in the thalamus and some leakage into surrounding structures, the step-to-mid ranged from 5.92 mm to 7.69 mm with mean distance of 7.18±0.27 mm, the step-to-ent ranged from 1.26 mm to 2.18 mm with mean distance of 1.79±0.19 mm in 4 cases with leakage into WMT, and the step-to-lat ranged from 1.33 mm to 1.88 mm with mean distance of 1.67±0.19 mm in 3 cases with leakage into Len. There were significant differences in step-ent and step-lat between excellent Vd group and good Vd with leakage group. The angle between cannula and horizontal line ranged from 61.08 to 69.89 degree with a mean 64.65±1.46 degree.

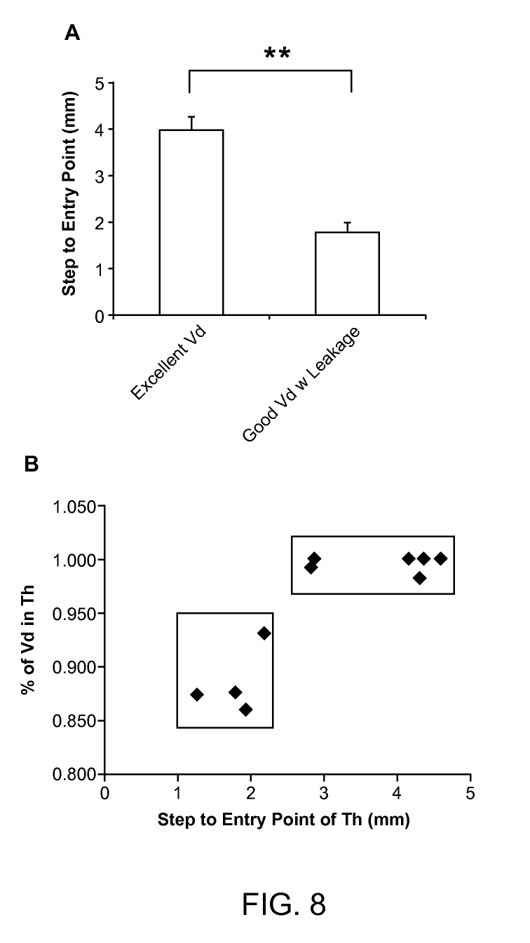

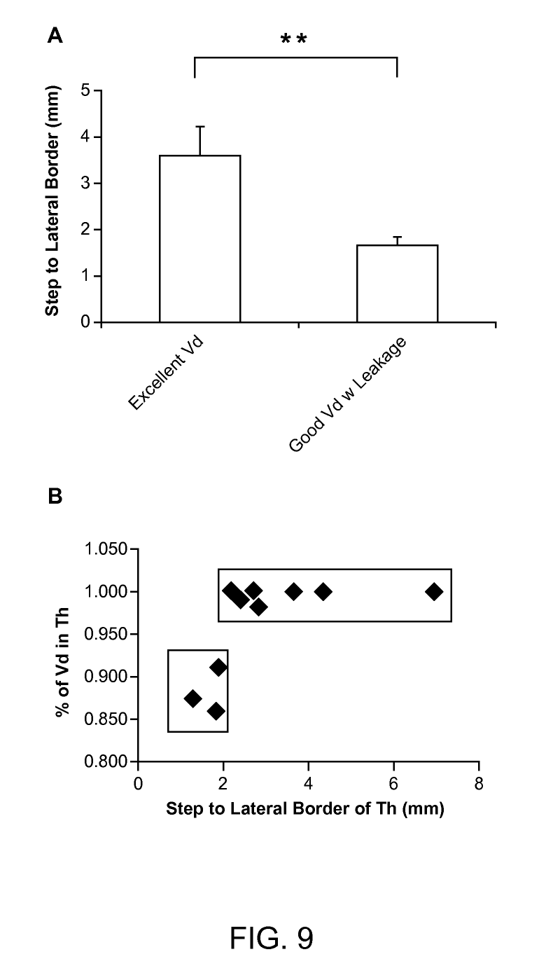

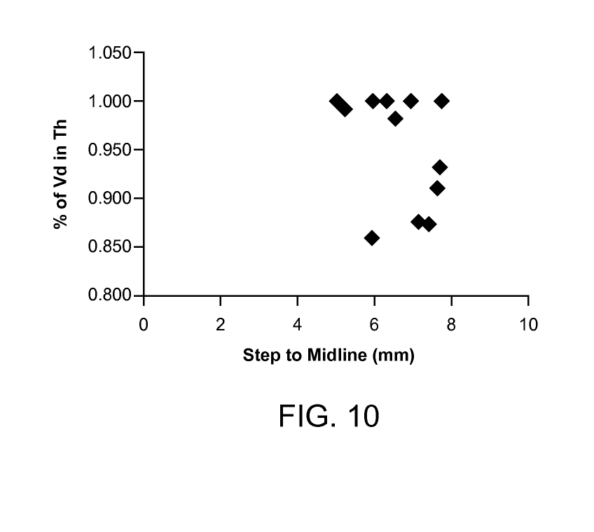

If the percent of infused tracer contained within the thalamus is plotted against each variable, it is apparent that distance from cannula step to its entry point or lateral border of thalamus correlates (FIGS. 8 and 9) with a sharp decline in distribution of infusate into the thalamus. In 4 infusions with leakage into MWT, the cannula step was placed close to cannula entry point of thalamus with mean distance of 1.79 mm (FIG. 8A). In 3 infusions with leakage into Lenf, the cannula step was placed close to lateral border of thalamus with mean distance of 1.67 mm (FIG. 9A). We conclude that the step-to-ent and step-to-lat distances should exceed about 2.8 and 2.2 mm, respectively, for optimal containment of infusate within thalamus. The distance from the cannula step to midline correlated poorly with putaminal containment (FIG. 10).

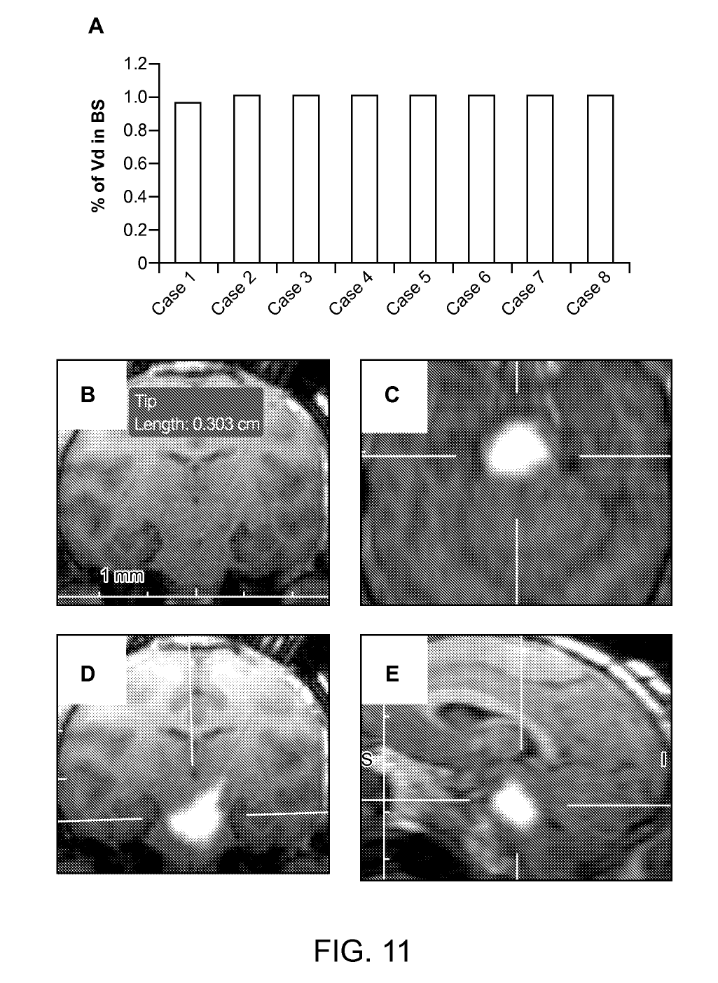

Distribution of Gadoteridol in the brainstem during CED. In all the 8 infusions (100%) performed in the brainstem, excellent distribution of Gd was achieved, and the Vd ranged from 224.3 to 886.3 mm³ with mean volume of 585.2±75.4 mm³. Only one case was found to have very few amount of leakage of Gd into thalamus at the end of infusion, and its Vd in thalamus was 30.5 mm³. The percent of Vd of Gd in the brainstem vs total Vd in brainstem and thalamus was 100% in 7 cases and 95.6% in one case (FIG. 11A). Infusion in the brainstem was well contained at infusion volume less than 212 μA used in this study. Brainstem infusion distributed rostrally towards mid-brain and caudal towards medulla oblongata. No distribution into cerebellum was seen. Representative MRIs show cannula step placement (FIG. 11B) and distribution of Gd (FIG. 11C to 11E) in the brainstem.

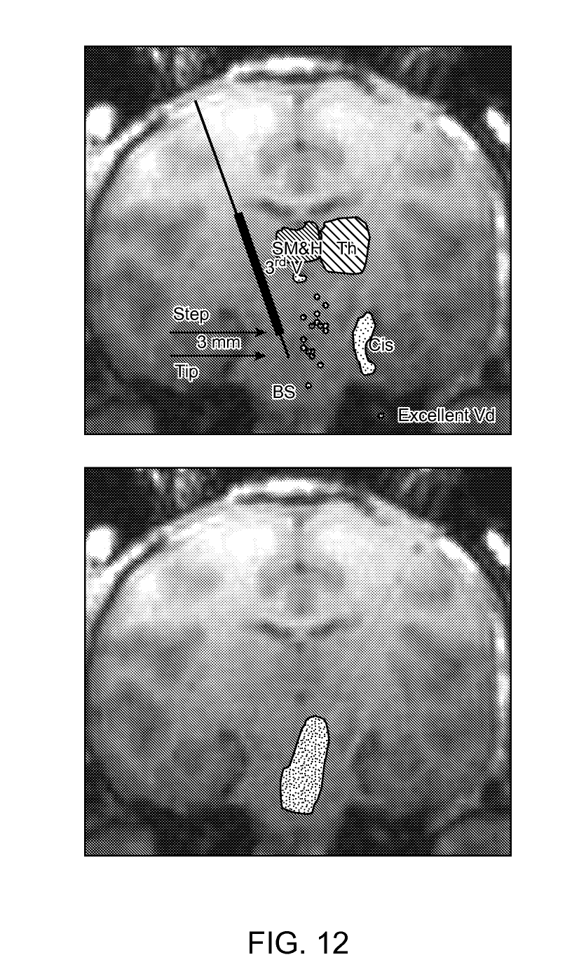

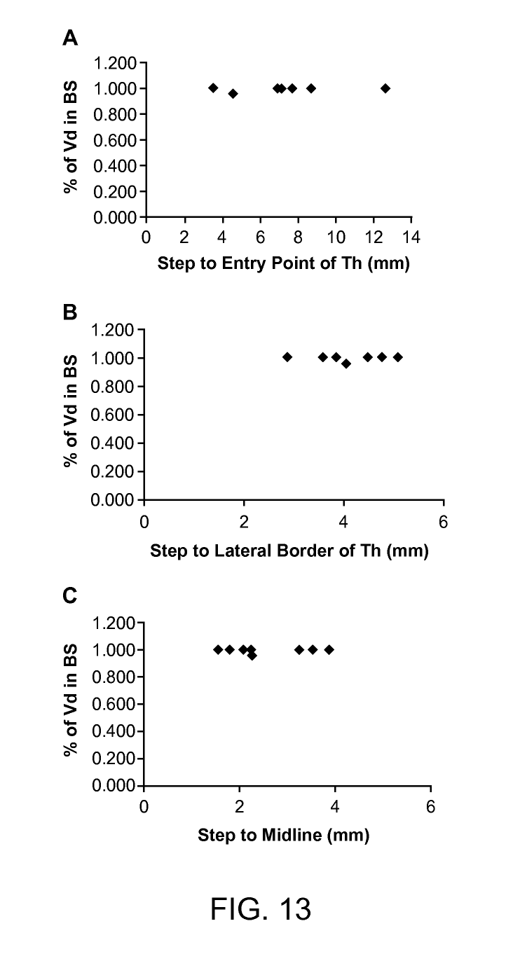

Measurements of parameters for cannula step placement in the brainstem. FIG. 12 shows the cannula placement in the brainstem in 8 cases with excellent distribution of Gd. The step-to-mid ranged from 1.56 mm to 3.88 mm with mean distance of 2.58±0.30 mm, the step-to-ent ranged from 3.55 mm to 12.63 mm with mean distance of 7.29±0.97 mm, and the step-to-lat ranged from 2.87 mm to 5.09 mm with mean distance of 4.14±0.25 mm. The angle between cannula and horizontal line ranged from 60.89 to 67.26 degree with a mean 64.27±0.83 degree. If the percent of infused tracer contained within the brainstem is plotted against each variable, it is apparent that cannula was placed appropriately so that optimal containment of infusate within brainstem was obtained (FIG. 13).

Three-dimensional reconstruction of volume of distribution of Gd in the thalamus and brainstem. Gd signal seen on MRI was outlined with BRainLab software, and 3D reconstruction of Vd was obtained in the thalamus (green) and brainstem (red). It shows the structured-related volume of distribution of Gd with robust distribution in the thalamus and brainstem. The volume of distribution in the thalamus and brainstem was plotted against volume of infusion (Vi). A linear trend line revealed a strong correlation between Vi and Vd in the thalamus in cases with excellent Vd (R²=0.997) and good Vd with leakage (R²=0.996) and in the brainstem (R²=0.992). According to these findings, a Vd three to four times as large as the Vi would be expected with Vi up to 158 μl in the thalamus and 212 μl in the brainstem. The over all Vd/Vi ratio of liposomes among structures infused in our study was 3.2 in thalamus and 3.9 in brainstem. Maximum distribution in the thalamus yielded around 660.3 mm³ for 158 μl, with distribution ratio of 417.9%, in the brainstem around 695.6 mm³ for 212 μl, with distribution ratio of 328.1%.

Green zones for cannula step in the thalamus and brainstem of NHP. On the basis of these analyses, we have defined coordinates for infusions in the thalamus and brainstem that identify preferred cannula characteristics and optimal distances from major structures in the brain.

When the cannula is placed in appropriate angle, the “green zone” in the thalamus is defined as at least 2.8 mm to entry point, greater than 2.2 mm from lateral border of thalamus, and more than 5 mm from midline. Similarly, when cannula is placed in appropriate angle, the “green zone” in the brainstem is defined as at least 3.5 mm to entry point, greater than 2.9 mm from lateral border of brainstem, and more than 1.6 mm from midline.

### Example 3

### MRI Predicts Distribution of GDNF in the NHP Brain after Convection-Enhanced Delivery of AAV2-GDNF

Gene therapies that utilize convention-enhanced delivery (CED) will require closely monitoring drug infusion in real time and accurately predicting drug distribution. Contrast (Gadoteridol, Gd) MRI was used to monitor CED infusion as well as to predict the expression pattern of therapeutic agent adeno-associated virus type 2 (AAV2) vector encoding glial cell line-derived neurotrophic factor (GDNF). The non-human primate (NHP) thalamus was utilized for modeling infusion to allow delivery of large clinically relevant volumes. Intracellular molecule AAV2 encoding aromatic L-amino acid decarboxylase (AADC) was co-infused with AAV2-GDNF/Gd to differentiate AAV2 transduction versus extracellular GDNF diffusion. The distribution volume of Gd (V_{d}) was linearly related to Vᵢ and the mean ratio of V_{d}/Vᵢ was 4.68±0.33. There was an excellent correlation between Gd distribution and AAV2-GDNF or AAV2-AADC expression and the ratios of expression areas of GDNF or AADC versus Gd were both close to 1. Our data support the use of contrast (Gd) MRI to monitor AAV2 infusion via CED and predict the distribution of AAV2 transduction.

The aim of the present study was to develop a method for enhanced safety and predictability in the delivery of AAV2-based gene therapy vectors to a target region. Specifically, this study is centered on a method of predicting AAV2-mediated GDNF expression volumes and patterns in the human striatum using co-infusion of the MRI tracer Gadoteridol (Gd, Prohance). Co-infusion of Gd and AAV2-GDNF allows near-real-time monitoring of infusions using repeated MRI T1 sequences. The development of an MRI-guided monitoring system is critical in translating our preclinical AAV2-GDNF gene therapy programs into clinical reality.

Preclinical studies of putaminal delivery of AAV2-GDNF via convection-enhanced delivery (CED) to aged and parkinsonian non-human primates (NHP) have proven that the putamen is the ideal delivery region for this gene therapy strategy. However, since the putamen of PD patients is approximately 5 times larger than the parkinsonian NHP putamen, infusion volume need to be scaled up to model the coverage required for the human putamen in clinical trials. The NHP putamen, however, can only be infused with volumes not exceeding 30-40 μL due to spillover of the infusate into the white matter tracts surrounding it. To better approximate infusion clinic parameters involved in maximizing coverage of the human putamen, we targeted the NHP thalamus, which is approximately 1.4 times the size of the NHP putamen but comparable to putamen in terms of proximity to surrounding structures. Thus, in the present study we infused AAV2-GDNF vector at clinically relevant volumes (˜150 μL) to the NHP thalamus to correlate patterns of Gd distribution with subsequent GDNF expression on the histological sections.

Previous studies have shown that intracerebral AAV2-GDNF infusion resulted in not only intracellular neuronal somata and fiber staining, but also extracellular immunoreactivity, suggesting that transduced GDNF protein is released into the extracellular space. This raises a possibility that extracellular GDNF protein may spread out through a concentration gradient-mediated diffusion. Thus the distribution of GDNF may be affected not only by AAV2 vector convection and transduction but possibly by extracellular GDNF protein diffusion as well. To better differentiate virus transduction versus GDNF protein diffusion, we co-infused a second AAV2 vector to express a non-secreted, intracellular molecule aromatic L-amino acid decarboxylase (AADC) with AAV2-GDNF/Gd. Since endogenous AADC is normally absent in the NHP thalamus, the expression of transduced AADC in the thalamus will provide reliable predictability on the boundary of AAV2 vector transduction and distribution.

### Materials and Methods

Experimental subjects and study design. Three normal adult NHP were the subjects in the present study. Experimentation was performed according to the National Institutes of Health guidelines and to the protocols approved by the Institute Animal Care and Use Committee at the University of California San Francisco (San Francisco, Calif.). The 3 NHP received intracranial infusions of AAV2 vectors and free gadoteridol (1 mM Gd, Prohance; Brancco Diagnostics, Princeton, N.J.) into the thalamus. Infusions were performed by previously established CED techniques for NHP.

Infusion formulation. Gadoteridol (Gd, C₁₇H₂₉N₄O₇Gd, Prohance) was purchased from Baracco Diagnostics Inc. (Princeton, N.J.). AAV2 vectors containing cDNA sequences for either human GDNF (AAV2-GDNF) or human AADC (AAV2-AADC) under the control of the cytomegalovirus promoter were packaged by the AAV Clinical Vector Core at Children's Hospital of Philadelphia using a triple-transfection technique with subsequent purification by CsCl gradient centrifugation. AAV2-GDNF/AAV2-AADC stock was concentrated to 2×10¹² vector genomes per ml (vg/ml) as determined by quantitative PCR, and then diluted immediately before use to 1˜1.2×10¹² vector genomes (vg/ml) in phosphate-buffered saline (PBS)-0.001% (v/v) Pluronic F-68.

Infusion procedure. NHP underwent neurosurgical procedures to position MRI-compatible guide arrays over the thalamus. Each customized guide array was cut to a specified length, stereotactically guided to its target through a burr-hole created in the skull and secured to the skull by dental acrylic. The larger diameter stem of the array had an outer and inner diameter of 0.53 and 0.45 mm, respectively. The outer and inner diameters of the tip segment were 0.436 and 0.324 mm, respectively. The tops of the guide array assemblies were capped with stylet screws for simple access during the infusion procedure. Animals recovered for at least 2 weeks before initiation of infusion procedures.

NHP were sedated with a mixture of ketamine (Ketaset, 7 mg/kg, IM) and xylazine (Rompun, 3 mg/kg, IM) and anesthetized with isoflurane (Aerrane; Ohmeda Pharmaceutical Products Division, Liberty Corner, N.J.). Each animal's head was placed in an MRI-compatible stereotactic frame, and a baseline MRI was performed before infusion to visualize anatomical landmarks and to generate stereotactic coordinates of the proposed target infusion sites for each animal. Vital signs, such as heart rate and PO2, were monitored throughout the procedure. Briefly, the infusion system consisted of a fused silica reflux-resistant cannula with a 3 mm step that was connected to a loading line (containing vectors and Gd), an infusion line with oil and another infusion line with trypan blue solution. A 1-ml syringe (filled trypan blue solution) mounted onto a micro-infusion pump (BeeHive; Bioanalytical System, West Lafayette, Ind.) regulated the flow of fluid through the system. Based on MRI coordinates, the cannula was manually guided to the targeted region of the brain through the previously placed guide array. The 3 mm step at the tip of the cannula to was designed to maximize fluid distribution during CED procedures and minimize reflux along the cannula tract. After securing placement of the infusion cannula. After securing placement of the infusion cannula, the CED procedures were initiated with real-time MRI data being acquired (real-time convective delivery, RCD). We used the same infusion parameters for every NHP infused throughout the study. Infusion rates were as follows: 1 μl/min was applied when lowering cannula to targeted area and increased at 2030-min intervals to 1.5 and 2.0 μl/min. After infusion, the cannula was withdrawn from the brain and the animals were allowed to recover under close observation until able to right themselves in their home cages.

Magnetic Resonance Image (MRI). MR images of brain were acquired on a 1.5-T Siemens Magnetom Avanto (Siemens AG, Munich, Germany). Three-dimensional rapid gradient echo (MP-RAGE) images were obtained with repetition time (TR)=17 ms, echo time (TE)=4.5 ms, flip angle=15°, number of excitations (NEX)=1 (repeated three times), matrix=256×256, field of view (FOV)=240×240×240 and slice thickness=1 mm. These parameters resulted in a 1-mm³ voxel volume. The scanning time was approximately 5 min per sequence with continuous scanning throughout the infusion procedure.

Volume and area quantification of Gd distribution from MR images. The volume of Gd distribution within each infused brain region was quantified with OsiriX Medical Image software (v.3.6). The software reads all data specifications from MR images. After the pixel threshold value for Gd signal is defined, the software calculates the signal above a defined threshold value, and establishes the area of region of interest (ROI) for each MRI series and computes the distribution volume V_{d} of ROI for the NHP brain. This allows V_{d} to be determined at any given time-point and can be reconstructed in a three-dimensional image.

Histological procedures. Animals were deeply anesthetized with sodium pentobarbital (25 mg/kg i.v.) and euthanized approximately 5 weeks after vector administration. The brains were harvested and coronally sliced with a brain matrix. The brain blocks were post fixed with 4% paraformaldehyde (PFA) and then cut into 40-μm coronal sections in a cryostat. Sections were processed for immunohistochemistry (IHC) staining. Serial sections were stained for glial derived neurotrophic factor (GDNF) and aromatic human I-amino acid decarboxylase (hAADC). Every 20th section was washed in phosphate buffered saline (PBS) and incubated in 1% H₂O₂ for 20 min to block the endogenous peroxidase activity. After washing in PBS, the sections were incubated in blocking solution Sniper® blocking solution (Biocare Medical, Concord, Calif.) for 30 min at RT followed by incubation with primary antibodies (GDNF, 1:500, R&D Systems, Minneapolis, Minn.; AADC, 1:1000, Chemicon, Billerica, Mass.; TH, 1:10000, Chemicon) in Da Vinci® diluent (Biocare Medical) overnight at RT. After 3 rinses in PBS for 5 min each at RT, sections were incubated in Mach 2 or Goat HRP polymer (Biocare Medical) for 1 h at RT, followed by several washes and colorimetric development (DAB; Vector Laboratories, Burlingame, Calif.; Vulcan Fast Red; Biocare Medical). Immunostained sections were mounted on slides and sealed with Cytoseal® (Richard-Allan Scientific, Kalamazoo, Mich.).

Area qualification of GDNF and AADC expression. The analysis of GDNF and AADC expression was performed with a Zeiss light microscope. GDNF- and AADC-positive areas were identified at low magnification and positively stained cells were confirmed under high magnification. Low magnification GDNF stained images were analyzed with ImageJ software and positively stained areas were identified with a threshold function. AADC-IR areas were outlined manually based on high magnification microscope imaging. Areas staining positive for GDNF or AADC were transferred to the corresponding primate MRI by manually delineating positive areas on the corresponding baseline MRI images using OsiriX software without reference to the MR images showing Gd distribution.

Statistical analysis. The areas of Gd distribution, GDNF or AADC expression were compared by Student's t-test and Pearson's correlation test. The criterion for statistical significance for all tests was p<0.05.

TABLE 2
Experimental design

| | Thalamus | |
|---|---|---|
| Primate | L side | R side |
| #1 | AAV2-GDNF/Gd | AAV2-GDNF/Gd |
| #2 | | AAV2-GDNF/AAV2-AADC/Gd |
| #3 | AAV2-GDNF/AAV2-AADC/ | AAV2-GDNF/AAV2-AADC/Gd |
| | Gd | |

### Results

Gd distribution in the thalamus. In this study, three rhesus primates were infused with ˜150 μL (Vᵢ) AAV2-GDNF/Gd (1˜1.2×10¹² vg/ml, n=5) to the thalamus; three of these infusions included AAV2-AADC (1×10¹² vg/ml, n=3) (Table 1). Magnetic resonance imaging (MRI) was performed before and during the infusion and coronal brain images every 1 cm apart were obtained to evaluate the distribution of Gd (V_{d}).

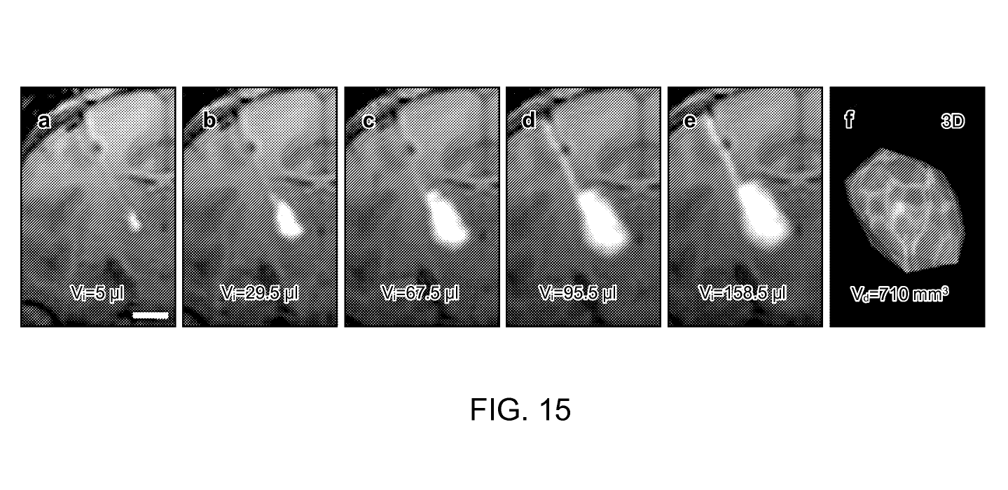

T1-weighted MRI was performed at 5-minute intervals and the images showed that the anatomical region with Gd infusion was clearly distinguishable from the surrounding non-infused tissue (FIG. 15a-15e). At the beginning of the infusion, a cylindrical ring of Gd distribution formed around the tip of the cannula (FIG. 15a). Infusion expanded radially to assume a more spherical pattern as the Vᵢ was increased (FIG. 15b-15e). 3D reconstructions of Gd distribution at the end of infusion with OsiriX software showed a tear-drop-shaped signal (FIG. 15f).

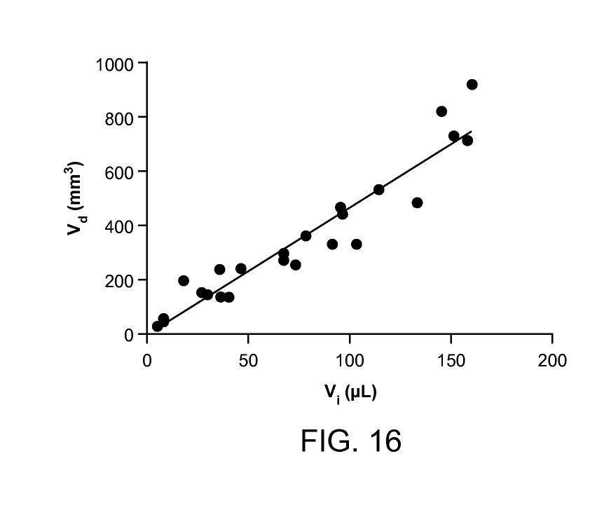

The volume of Gd distribution (V_{d}) at various time points was quantified with OsiriX software. Consistent with the gross MR imaging appearance during infusions (FIG. 15a-15e), the V_{d} of Gd increased linearly with Vᵢ (R²=0.904, P<0.0001) (FIG. 16), and the final volume ranged from 700 to 900 mm³, which covered approximately 70 to 90% of the total volume of the NHP thalamus. The ratio of V_{d}/Vᵢ for each infusion site remained consistent and the mean value was 4.68±0.33.

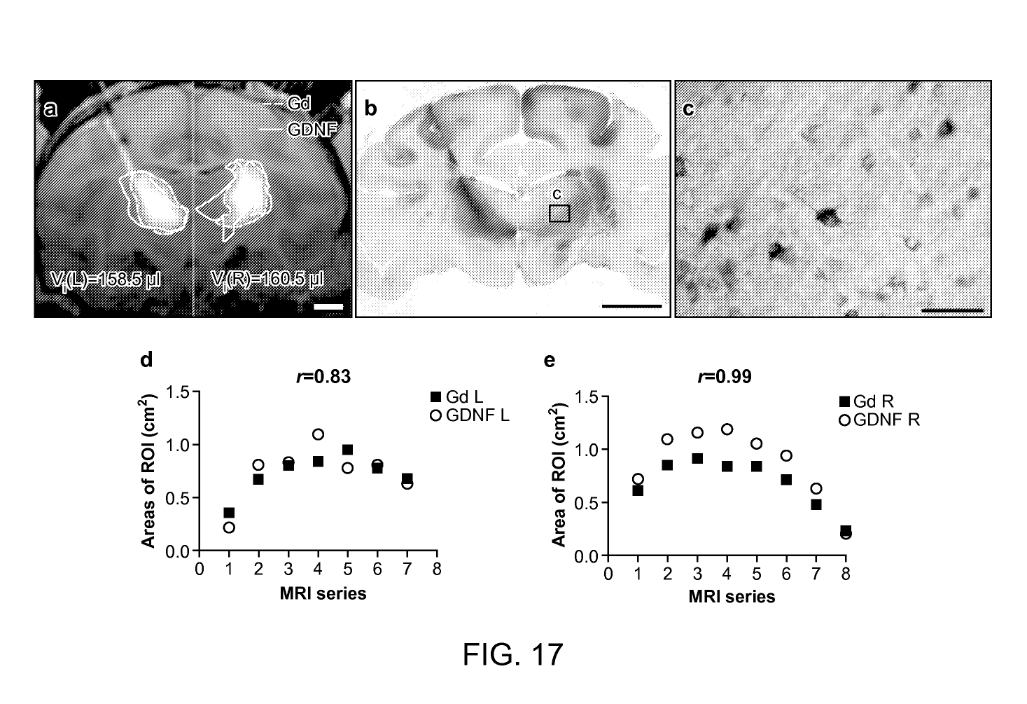

Correlation of Gd with GDNF histology. Animals were euthanized after 5 weeks and brain blocks containing the thalamus were post-fixed and sectioned coronally. Sets of serial sections 0.8 mm apart were stained with an antibody against GDNF. Immunohistochemical analysis demonstrated that the expression pattern of GDNF protein in the infusion site was similar to Gd distribution (FIGS. 17a and 17b). A quantitative analysis showed that the areas of GDNF expression were highly correlated with those of Gd distribution (FIGS. 17d and 17e). The average ratio of GDNF staining areas vs. Gd distribution areas was 1.08±0.17. High magnification microscopy images showed that GDNF staining was observed in the cytoplasm of neuronal cells as well as in extracellular space with a staining pattern suggestive of GDNF binding to extracellular matrix (FIG. 17c).

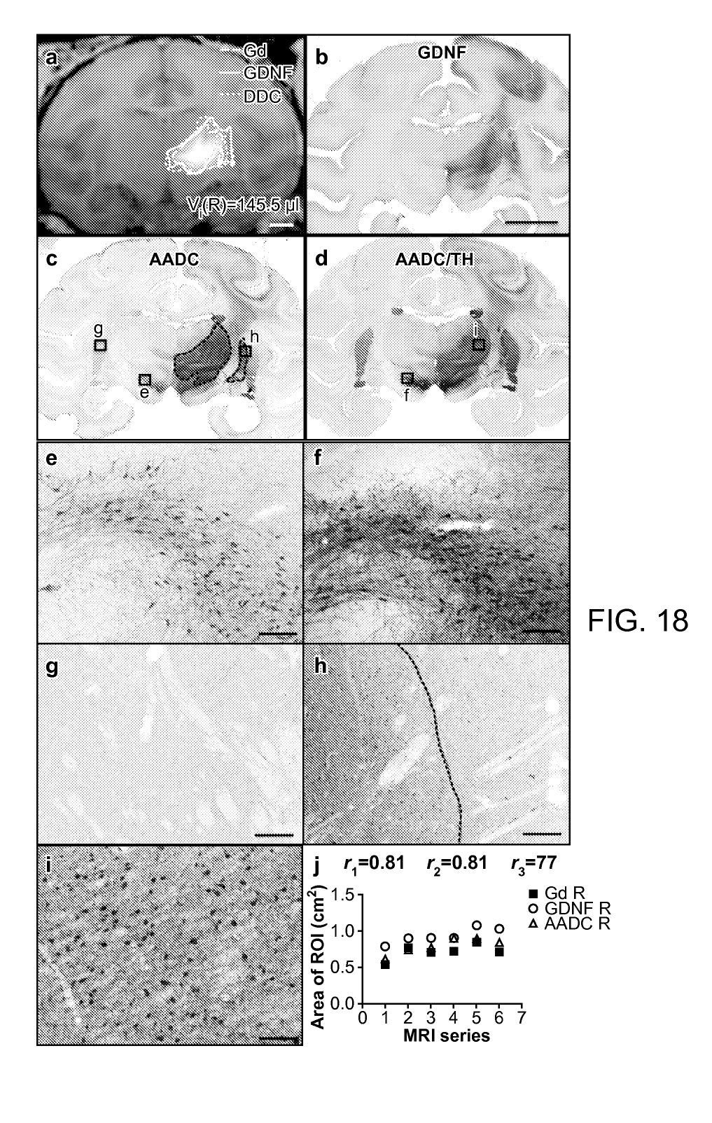

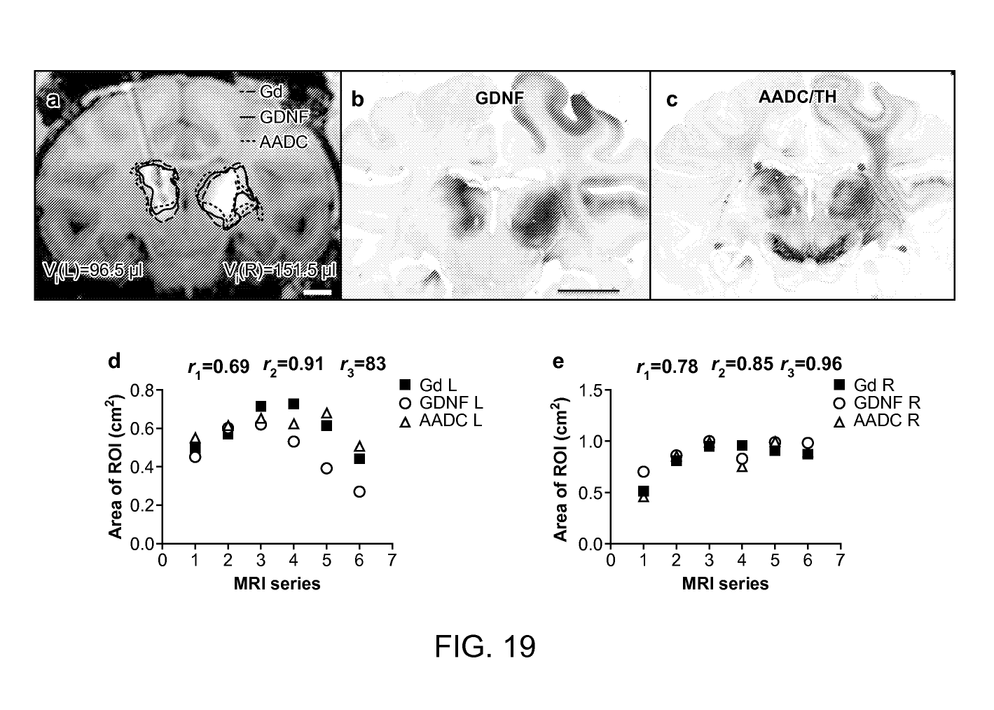

Robust GDNF staining was observed in distinct cortical regions, far from the needle tract, in all animals after thalamic AAV2-GDNF infusion (FIGS. 17b, 18b and 19b). We also found AADC staining in the cortex of NHP co-infused with AAV2-AADC (FIGS. 18c and 19c). The presence of GDNF or AADC protein in the cortex was due to the axonal transport from the thalamus. Thus, in the current study we excluded the staining in thalamo-cortical fibers and cortex from measured areas of gene expression, to better compare Gd distribution with GDNF or AADC expression that was derived primarily from direct convective delivery within the thalamus.

Correlation of GDNF and AADC histology. Thalamic delivery of AAV2-GDNF resulted in robust intracellular and extracellular GDNF immunoreactivity. Given the broad distribution of MRI tracer Gd, the considerable GDNF distribution in the present study can be attributed to dispersion of the volume of infused vector (˜150 μL). However, levels of extracellular diffusion of GDNF may affect distribution as well. Thus, in order to assess the effect of extracellular diffusion on the total area of gene expression, areas of GDNF expression were compared to areas of intracellular molecule AADC expression in animals with co-infusion of AAV2-AADC. In this way, cell transduction versus secretion and diffusion of the gene product could be differentiated.

Two primates (#2 and #3) were co-infused with AAV2-GDNF and AAV2-AADC into the thalamus; one received unilateral infusion and the other one received bilateral. Adjacent brain sections containing thalamic infusions were stained for GDNF and AADC respectively. In addition, since AADC immunostaining can detect both transduced and endogenous AADC in the NHP (FIGS. 18c and 18e), we developed a double chromogenic staining method to differentiate transduced AADC from endogenous AADC which was co-localized with TH-positive profiles. Sections were dual labeled for AADC in light brown and endogenous tyrosine hydroxylase (TH) in bright red (FIG. 18d). Nearly all neurons that contained endogenous AADC were positive for TH. Thus, cells containing endogenous AADC as well as TH were double-labeled and stained in dark red (FIG. 18f) and only those transduced neurons with exogenous AADC was stained with the single chromagen and appeared light brown (FIG. 18i). By superposing the adjacent AADC stained sections with AADC/TH dual stained sections, we were able to delineate the boundary of transduced AADC expression (FIG. 18c, blue line).

The unilateral co-infusion of AAV2-GDNF and AAV2-AADC into the thalamus of one primate (#2) allowed easy differentiation of endogenous and transduced AADC, since transduced AADC was only observed on the infused side of the brain. In contrast, endogenous AADC, which was colocalized with TH, was present bilaterally in the caudate, putamen and substantia nigra (FIG. 18d). For this particular primate, as the thalamic infusion extended to the medial aspect of putamen, AADC positive cells were found at the edge of medial putamen (FIG. 18h), in contrast to the left putamen which contained only endogenous AADC positive fibers (FIG. 18g). These AADC positive cells in the right putamen were included for area measurements as outlined in blue (FIG. 18h). The overall AADC staining intensity in the right putamen and caudate appeared greater compared to the left side (FIGS. 18c, 18g and 18h). We also observed a similar pattern in the GDNF staining sections (FIG. 18b). The enhanced immunoactivity of AADC or GDNF on the right putamen and caudate was most likely due to the anterograde transportation of expressed gene product from the dorsal nigra where infusion extended in this primate. Thus these regions were not included as direct vector transduction areas.

By comparing the adjacent GDNF and AADC/TH stained sections, we saw that the expression patterns of GDNF and exogenous AADC in the thalamus were nearly identical. In addition, GDNF and AADC expression substantially overlapped with MRI Gd distribution (FIG. 18a). The areas of Gd, GDNF and AADC distribution in a series of MRI coronal planes were highly correlated with one another (FIG. 18j). The average ratio of AADC staining areas vs. Gd distribution areas was 1.07±0.06, which is equivalent to GDNF vs. Gd (1.08±0.17). All of these data strongly indicated an excellent match between AADC and GDNF distribution.

Bilateral co-infusion of AAV2-GDFN and AAV2-AADC into the thalamus of the other primate (#3) further validated our findings (FIG. 19). The majority of transduced GDNF and AADC protein were confined to both sides of thalamus (FIGS. 19b and 19c), where expression patterns were highly correlated with Gd distribution (FIGS. 19a, 19d and 19e).

In the present study, we used an MRI contrast agent to visualize the infusion in near-real-time in order to predict the distribution of a therapeutic agent AAV2-GDNF. The NHP thalamus was utilized for modeling infusions in the human putamen to allow delivery of clinically relevant volumes. We were able to administer vector at a Vᵢ of ˜150 μL into the thalamus by CED without reflux or leakage. V_{d} of Gd was linearly related to Vᵢ and the mean ratio of V_{d}/Vᵢ was 4.68±0.33. There was an excellent correlation between Gd distribution and both AAV2-GDNF and AAV2-AADC expression and the ratios of expression areas of GDNF or AADC versus Gd were both close to 1, strongly suggesting that we can predict the distribution of AAV2 transduction and subsequent gene expression with contrast (Gd) MRI. In addition, since the expression patterns of GDNF and AADC are nearly identical, there was no detectable diffusion of GDNF protein after AAV2-GDNF transduction. Thus, anticipated GDNF expression in the patients who receive AAV2-GDNF in future clinical trials can be expected to be approximately 4-5-fold larger than Vᵢ of co-infused Gd, without any additional coverage due to diffusion of GDNF from the transduced region. This information is critical for accurately selecting the dose of AAV2-GDNF vector for clinical studies.

Intracerebral infusion of powerful therapies directly into disease-affected regions using CED provides an effective strategy for treating neurological disorders. In the current study, co-infusion of MRI contrast enhancement agent Gd with therapy AAV2-GDNF using CED proved to be useful in monitoring infusion and estimating therapy distribution. Real-time MR imaging with Gd revealed an infusion region that was easily distinguishable from surrounding tissue (FIG. 15A-15E). This well-defined infusion region allowed for near-real-time adjustment of infusion parameters and precise volumetric analysis.

During CED infusion, the difference in distribution between Gd and AAV vector is rather minor, probably due to the predominant driving force of pressure gradient-mediated fluid advection rather than concentration gradient-mediated diffusion. Thus, MRI Gd signal can reliably mimic the distribution of AAV2 vector during infusion. For longer time scales after infusion finishes, the distribution of AAV2 vector as well as extracellular GDNF released by transduced cells in the brain may solely depend on the concentration gradient and the diffusivity of the infusate in the tissue. We found that the distribution of Gd based on near-real-time MRI during infusion was highly correlated with GDNF expression 5 weeks after infusion and the ratio for Gd vs GDNF was close to 1. Furthermore, the distribution of GDNF was nearly identical to the intracellular molecule AADC. These findings were in consistent with previous studies and strongly suggested limited diffusion of AAV2 vector or GDNF after the infusion stopped. Therefore the distribution of CED infusion of Gd may effectively predict the distribution of AAV2-GDNF both acutely and over longer time periods.

The distribution of Gd (V_{d}) increased linearly with the volume of infusate (Vᵢ) and the ratio for V_{d} to Vᵢ was 4.68±0.33, which is within the relatively narrow range of previous work (approximately 4˜5;). This constant linear relationship of V_{d} with Vᵢ in the MRI-guided CED delivery platform may allow a foundation for planning clinical doses of AAV2-GDNF vector as well as prediction of the distribution of this and other therapeutic agents in patients with PD.

In summary, we are able to infuse AAV2-GDNF vector accurately to the targeted brain region via CED using near-real-time MRI imaging. Contrast MRI additionally provides a valuable tool to guide AAV2 vector infusion and predict AAV2-GDNF expression reliably, allowing for increased safety, precision and clinically-relevant coverage of the putamen with this vector in PD patients.

1. A method of delivering a therapeutic agent to a targeted region of a primate brain, the method comprising:
selecting a position for the cannula insertion, wherein the tip position is at least about 1 mm distant from a leakage pathway; and
delivering said therapeutic agent through said delivery cannula to said targeted region.

2. The method according to claim 1, wherein the therapeutic agent is delivered by convection-enhanced delivery.

3. The method according to claim 2, wherein the delivery cannula is a reflux-resistant step cannula.

4. The method of claim 1, wherein the primate is a non-human primate.

5. The method of claim 1, wherein the primate is a human.

6. The method of claim 4, wherein the targeted region of the brain is within the cerebrum.

7. The method of claim 6, wherein the placement of the delivery cannula is selected to be at least about 2 mm from a leakage pathway.

8. The method of claim 6, wherein the placement of the delivery cannula is selected to be at least about 3 mm from a leakage pathway.

9. The method of claim 8, wherein the leakage pathway is an axon tract selected from the corpus callosum (CC), anterior commissure (AC); external capsule (EC), and internal capsule (IC).

10. The method of claim 6, wherein the targeted region of the brain is selected from striatum, caudate, putamen, globus pallidus, nucleus accumbens; septal nuclei, and subthalamic nucleus.

11. The method of claim 10, wherein the targeted region is the putamen.

12. The method of claim 4, wherein the targeted region of the brain is the thalamus or hypothalamus.

13. The method of claim 12, wherein the placement of the delivery cannula tip is selected to be at least 2.5 mm from the entry point; at least 1.8 mm from the lateral border; and at least 4.5 mm from midline.

14. The method of claim 12, wherein the placement of the delivery cannula tip is selected to be at least 3 mm from the entry point; at least 2.2 mm from the lateral border; and at least 5 mm from midline.

15. The method of claim 4, wherein the targeted region of the brain is within the brainstem.

16. The method of claim 15, wherein the placement of the delivery cannula tip is selected to be at least 2.8 mm from the entry point; at least 2.5 mm from the lateral border; and at least 1.25 mm from midline.

17. The method of claim 15, wherein the placement of the delivery cannula tip is selected to be at least 3.5 mm from the entry point; at least 2.92 mm from the lateral border; and at least 1.6 mm from midline.

18. The method of claim 17, wherein the targeted region is selected from substantia nigra, red nucleus, pons, olivary nuclei, and cranial nerve nuclei.

19. A method of treating a central nervous system disorder, the method comprising administering a therapeutic agent by the method set forth in claim 1.

20. A system for delivery of therapeutic agents to a primate brain, where the system comprises a stereotactic system for positioning a cannula at least about 1 mm distant from a leakage pathway, and wherein the stereotactic system comprises a set of coordinates for positioning a delivery cannula within a previously defined zone determined to provide quantitative containment of infusate in said targeted region for the primate.

21. The system of claim 21, further comprising a delivery cannula.

22. The system of claim 21, wherein the therapeutic agent is delivered by convection-enhanced delivery.

23. The system of claim 22, wherein the delivery cannula is a reflux-resistant step cannula.

24. A method of determining a green zone in a targeted region of a primate brain for delivery cannula positioning, wherein a delivery cannula positioned within the green zone provides quantitative containment of infusate in said targeted region, the method comprising:
delivering an imaging agent to the targeted region of the brain through a delivery cannula;
determining the distribution of infused imaging agent; and
correlating the site of delivery cannula placement with the desired distribution, wherein the set of coordinates for optimal placement are those that result in appropriately contained infusate.

25. The method of 24, further comprising:
determining by 3-dimensional modeling a green zone in a different primate species for said targeted region of the brain.
